SEQUENCE LISTING (12 sequences)

SEQ ID NO: 1 - protein, 1273 aa, SARS-CoV-1
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 419 aa, SARS-CoV-1
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 3 - protein, 222 aa, SARS-CoV-1
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 4 - protein, 75 aa, SARS-CoV-1
MYSFVSEETGTLIVNSVLLFLAFVVFLLTVLAILTALRLCAYCCNIVNVSLVKPSFYVYSRVKNLNSSRVPDLLS

SEQ ID NO: 5 - protein, 75 aa, SARS-CoV-1
MYSFVSEETGTLIVNSVLLFLAFVVFLLVTLAILTALRLCAYCCNIVNVSLVKPSFYVYSRVKNLNSSRVPDLLV

SEQ ID NO: 6 - protein, 275 aa, SARS-CoV-1
MDLFMRIFTIGTVTLKQGEIKDATPSDFVRATATIPIQASLPFGWLIVGVALLAVFQSASKIITLKKRWQLALSKGVHFVCNLLLLFVTVYSHLLLVAAGLEAPFLYLYALVYFLQSINFVRIIMRLWLCWKCRSKNPLLYDANYFLCWHTNCYDYCIPYNSVTSSIVITSGDGTTSPISEHDYQIGGYTEKWESGVKDCVVLHSYFTSDYYQLYSTQLSTDTGVEHVTFFIYNKIVDEPEEHVQIHTIDGSSGVVNPVMEPIYDEPTTTTSVPL

SEQ ID NO: 7 - protein, 61 aa, SARS-CoV-1
MFHLVDFQVTIAEILLIIMRTFKVSIWNLDYIINLIIKNLSKSLTENKYSQLDEEQPMEID

SEQ ID NO: 8 - protein, 38 aa, SARS-CoV-1
MGYINVFAFPFTIYSLLLCRMNSRNYIAQVDVVNFNLT

SEQ ID NO: 9 - protein, 97 aa, SARS-CoV-1
MDPKISEMHPALRLVDPQIQLAVTRMENAVGRDQNNVGPKVYPIILRLGSPLSLNMARKTLNSLEDKAFQLTPIAVQMTKLATTEELPDEFVVVTVK

SEQ ID NO: 10 - protein, 121 aa, SARS-CoV-1
MKIILFLALITLATCELYHYQECVRGTTVLLKEPCSSGTYEGNSPFHPLADNKFALTCFSTQFAFACPDGVKHVYQLRARSVSPKLFIRQEEVQELYSPIFLIVAAIVFITLCFTLKRKTE

SEQ ID NO: 11 - protein, 43 aa, SARS-CoV-1
MIELSLIDFYLCFLAFLLFLVLIMLIIFWFSLELQDHNETCHA

SEQ ID NO: 12 - protein, 121 aa, SARS-CoV-2
MKFLVFLGIITTVAAFHQECSLQSCTQHQPYVVDDPCPIHFYSKWYIRVGARKSAPLIELCVDEAGSKSPIQYIDIGNYTVSCLPFTINCQEPKLGSLVVRCSFYEDFLEYHDVRVVLDFI

(19) United States
(12) Patent Application Publication
(10) Pub. No.: US 2023/0349889 A1
(43) Pub. Date: Nov. 2, 2023

(54) Methods of Determining Immune Response

(71) Applicant: The Council of the Queensland Institute of Medical Research, Queensland (AU)

(72) Inventors: Corey Smith, Queensland (AU); Rajiv Khana, Queensland (AU); Katie Lineburg, Queensland (AU)

(21) Appl. No.: 18/025,763

(22) PCT Filed: Sep. 10, 2021
(86) PCT No.: PCT/AU2021/051051

§ 371 Date: Mar. 10, 2023

(30) Foreign Application Priority Data
Sep. 10, 2020 (AU) 2020903246

(51) Int. Cl. G01N 33/50; G01N 33/68

(52) U.S. Cl. CPC G01N 33/5091; G01N 33/6869; G01N 2333/5421; G01N 2333/165; G01N 2333/55; G01N 2333/57; G01N 2800/24

(57) ABSTRACT

The present disclosure relates generally to the field of immunological-based assays and describes methods for measuring cell-mediated immuno responsiveness. More particularly, the present disclosure relates to methods, compositions, and kits for measuring cell-mediated immune response activity with enhanced sensitivity and improved storage stability.

### RELATED APPLICATIONS

This application claims priority to Australian Provisional Application No. 2020 entitled “Methods of Determining Immune Response” filed 10 Sep. 2020, the contents of which are incorporated herein by reference in their entirety.

### FIELD OF THE DISCLOSURE

This disclosure relates generally to the field of immunological-based assays and describes methods for measuring cell-mediated immunoresponsiveness. The present disclosure teaches methods, compositions, and kits for measuring cell-mediated immune response activity with enhanced sensitivity and improved storage stability.

### BACKGROUND OF THE INVENTION

In less than two years, as of September 2021, the SARS-CoV-2 virus has infected more than 200,000,000 individuals worldwide and caused more than four million deaths. Despite optimism around effective vaccines that have been developed in the last 12 months, and that the therapeutic administration of anti-viral drugs will reduce the severity of symptoms associated with Covid-19, it is likely that millions more individuals will be infected over the coming years. Therefore, developing an understanding of all aspects of immunity to SARS-CoV-2 is critical. This is particularly so in light of recent reports that the protection provided by the current vaccines diminishes with time, and booster shots will likely be required in order to ensure that at-risk individuals retain suitable immune responsivity. Antigen-specific T cells that can recognise viral proteins in the context of MHC class I and II molecules on the surface of infected cells are critical mediators of the cellular immune response to all viral pathogens [1, 2]. These T cells have a myriad of functions that act to both augment other immune responses and that directly target viral infected cells, as in T cell mediated cytolysis of infected cells.

As a consequence of their significant role in immunological help, virus-specific T cell responses are typically established early after primary infection. However, studies over the past two decades have shown that poor induction of the T-cell response can lead to significant T cell dysfunction and chronic infection. Early observations in patients with Covid-19 provide evidence for T cell dysfunction as a cause of disease development. Patients with severe symptoms show high expression of checkpoint markers, including PD-1 suggesting poor T cell priming during the early stages of infection. Conversely, while only preliminary analysis has been performed, patients who resolve infection have recently been shown to generate antigen specific T cell responses against proteins encoded by SARS-CoV-2. These observations support the contention that T cells play a critical role in the immune response to SARS-CoV-2 infection. They also suggest that the monitoring of antigen-specific T cell response during infection could provide a key attribute to determining risk of serious Covid-19 disease, and suggest that immune interventions, such as vaccines, should also target T cell immunity to reduce disease risk.

### SUMMARY OF THE INVENTION

The present invention was predicated, at least in part, on the determination by the present inventors that a biological sample comprising T cells could be used to determine the immunocompetence of an individual to a SARS-CoV virus (e.g., SARS-CoV-1 and SARS-CoV-2). More particularly, it was determined that peptide antigens corresponding to nucleoprotein, spike protein, and/or membrane protein could be used to elicit a T cell response in a sample obtained from a subject that had previously been exposed to a SARS-CoV-2.

In one aspect, the present invention provides methods of determining cell-mediated immune response activity, the method comprising: contacting a sample comprising immune cells capable of producing immune effector molecules following stimulation, with one or more peptide antigens corresponding to at least one coronavirus protein; and detecting the presence or the level of an immune effector molecule.

In some embodiments the immune effector molecule is selected from IL-2, IFN-γ, and IL-8. In some preferred embodiments, the immune effector molecule is IL-2. In some of the same embodiments, the immune effector molecule also includes one or both of IFN-γ and IL-8.

In some preferred embodiments, the sample is a whole blood sample. In some alternative embodiments, the sample is a PBMC sample. In some embodiments, the immune cells are selected from the group comprising or consisting of T cells, B cells, dendritic cells, macrophages, monocytes, or NK cells.

Typically, the sample comprises at least one T cell (e.g., a CD8⁺ T cell and/or a CD4⁺ T cell). Preferably, the sample comprises at least one T cell which is a CD8⁺ cytotoxic T cell.

In some embodiments, the plurality of antigens are formulated as a single composition. The composition may be suitably comprised in a sample collection vessel, such that the sample is exposed to the composition on entering the vessel.

In some embodiments, and particularly those wherein the sample is a whole blood sample, the sample is exposed to an anticoagulant. In some preferred embodiments of this type, the anticoagulant is heparin.

Typically, the sample is obtained from a human subject.

In some embodiments, the sample is immunosuppressed or immunodeficient. In some embodiments, the plurality of antigens comprise peptides that correspond to at least part of a coronavirus protein (e.g., a SARS-CoV-2 protein). Typically, the peptides each have a length selected from 5 to 100 amino acids or 7 to 50 amino acids.

In some embodiments, the plurality of antigens correspond to at least 2, 3, 4, 5, 6, 7, 8, 9, 10, 11, 12, 13, 14, 15, 16, 17, 18, 19, 20, or more than 20 coronavirus proteins. In some alternative embodiments, the plurality of peptides correspond to a single coronavirus (e.g., SARS-CoV-2) protein.

In some embodiments at least some of the plurality of antigens correspond to, and therefore are cross-reactive with, a structural protein from a coronavirus (e.g., SARS-CoV-2).

In some embodiments the coronavirus proteins are selected from the group comprising: nucleoprotein, membrane protein, spike glycoprotein, protein 3a, non-structural protein 6, ORF10 protein, ORGF9B protein, non-structural protein 7a, non-structural protein 7b, non-structural protein 8, uncharacterised protein 14, and envelope small membrane protein.

In some embodiments, the plurality of antigens comprise one or more peptides with an amino acid sequence that corresponds to at least a portion of spike glycoprotein.

In some preferred embodiments, the spike glycoprotein is the wild-type SARS-CoV-2 spike glycoprotein with the UniProtKB accession no. PODTC2, and set forth below:

[SEQ ID NO: 2]
MFVFLVLLPLVSSQCVNLTTRTQLPPAYTNSFTRG
VYYPDKVFRSSVLHSTQDLFLPFFSNVTWFHAIHV
SGTNGTKRFDNPVLPFNDGVYFASTEKSNIIRGWI
FGTTLDSKTQSLLIVNNATNVVIKVCEFQFCNDPF
LGVYYHKNNKSWMESEFRVYSSANNCTFEYVSQPF
LMDLEGKQGNFKNLREFVFKNIDGYFKIYSKHTPI
NLVRDLPQGFSALEPLVDLPIGINITRFQTLLALH
RSYLTPGDSSSGWTAGAAAYYVGYLQPRTFLLKYN
ENGTITDAVDCALDPLSETKCTLKSFTVEKGIYQT
SNFRVQPTESIVRFPNITNLCPFGEVFNATRFASV
YAWNRKRISNCVADYSVLYNSASFSTFKCYGVSPT
KLNDLCFTNVYADSFVIRGDEVRQIAPGQTGKIAD
YNYKLPDDFTGCVIAWNSNNLDSKVGGNYNYLYRL
FRKSNLKPFERDISTEIYQAGSTPCNGVEGFNCYF
PLQSYGFQPTNGVGYQPYRVVVLSFELLHAPATVC
GPKKSTNLVKNKCVNFNFNGLTGTGVLTESNKKFL
PFQQFGRDIADTTDAVRDPQTLEILDITPCSFGGV
SVITPGTNTSNQVAVLYQDVNCTEVPVAIHADQLT
PTWRVYSTGSNVFQTRAGCLIGAEHVNNSYECDIP
IGAGICASYQTQTNSPRRARSVASQSIIAYTMSLG
AENSVAYSNNSIAIPTNFTISVTTEILPVSMTKTS
VDCTMYICGDSTECSNLLLQYGSFCTQLNRALTGI
AVEQDKNTQEVFAQVKQIYKTPPIKDFGGFNFSQI
LPDPSKPSKRSFIEDLLFNKVTLADAGFIKQYGDC
LGDIAARDLICAQKFNGLTVLPPLLTDEMIAQYTS
ALLAGTITSGWTFGAGAALQIPFAMQMAYRFNGIG
VTQNVLYENQKLIANQFNSAIGKIQDSLSSTASAL
GKLQDVVNQNAQALNTLVKQLSSNFGAISSVLNDI
LSRLDKVEAEVQIDRLITGRLQSLQTYVTQQLIRA
AEIRASANLAATKMSECVLGQSKRVDFCGKGYHLM
SFPQSAPHGVVFLHVTYVPAQEKNFTTAPAICHDG
KAHFPREGVFVSNGTHWFVTQRNFYEPQIITTDNT
FVSGNCDVVIGIVNNTVYDPLQPELDSFKEELDKY
FKNHTSPDVDLGDISGINASVVNIQKEIDRLNEVA
KNLNESLIDLQELGKYEQYIKWPWYIWLGFIAGLI
AIVMVTIMLCCMTSCCSCLKGCCSCGSCCKFDEDD
SEPVLKGVKLHYT.

Alternatively, the spike protein may be a natural variant of the amino acid sequence presented above.

In some embodiments, the plurality of antigens comprise one or more peptides with an amino acid sequence that corresponds to at least a portion of the SARS-CoV-2 nucleoprotein.

In some preferred embodiments, the nucleoprotein is the wild-type SARS-CoV-2 nucleoprotein with the UniProtKB accession no. PODTC9, and set forth below:

[SEQ ID NO: 2]
MSDNGPQNQRNAPRITFGGPSDSTGSNQNGERSGA
RSKQRRPQGLPNNTASWFTALTQHGKEDLKFPRGQ
GVPINTNSSPDDQIGYYRRATRRIRGGDGKMKDLS
PRWYFYYLGTGPEAGLPYGANKDGIIWVATEGALN
TPKDHIGTRNPANNAAIVLQLPQGTTLPKGFYAEG
SRGGSQASSRSSSRSRNSSRNSTPGSSRGTSPARM
AGNGGDAALALLLLDRLNQLESKMSGKGQQQQGQT
VTKKSAAEASKKPRQKRTATKAYNVTQAFGRRGPE
QTQGNFGDQELIRQGTDYKHWPQIAQFAPSASAFF
GMSRIGMEVTPSGTWLTYTGAIKLDDKDPNFKDQV
ILLNKHIDAYKTFPPTEPKKDKKKKADETQALPQR
QKKQQTVTLLPAADLDDFSKOLQQSMSSADSTQA.

Alternatively, the nucleoprotein may be a natural variant of the amino acid sequence presented above.

In some alternative embodiments, the plurality of antigens comprise a peptide with an amino acid sequence that corresponds to at least a portion of spike glycoprotein and a peptide with an amino acid sequence that corresponds to at least a portion of nucleoprotein.

In some alternative embodiments, the plurality of antigens comprise a peptide with an amino acid sequence that corresponds to at least a portion of spike glycoprotein and a peptide with an amino acid sequence that corresponds to at least a portion of membrane protein.

In some preferred embodiments, the membrane protein is the wild-type SARS-CoV-2 membrane protein with the UniProtKB accession no. PODTC5, and amino acid sequence set forth below:

[SEQ ID NO: 3]
MADSNGTITVEELKKLLEQWNLVIGFLFLTWICLL
QFAYANRNRFLYIIKLIFLWLLWPVTLACFVLAAV
YRINWITGGIAIAMACLVGLMWLSYFIASFRLFAR
TRSMWSFNPETNILLNVPLHGTILTRPLLESELVI
GAVILRGHLRIAGHHLGRCDIKDLPKEITVATSRT
LSYYKLGASQRVAGDSGFAAYSRYRIGNYKLNTDH
SSSSDNIALLVQ.

Alternatively, the membrane protein may be a natural variant of the amino acid sequence presented above.

In some alternative embodiments, the plurality of antigens comprise a peptide that corresponds to at least a portion of spike glycoprotein and a peptide with an amino acid sequence that corresponds to at least a portion of envelope small membrane protein.

In some preferred embodiments, the envelope small membrane protein is the SARS-CoV-2 envelope small membrane protein with the amino acid sequence set forth below:

[SEQ ID NO: 4]
MYSFVSEETGTLIVNSVLLFLAFVVFLLTVLAILT
ALRLCAYCCNIVNVSLVKPSFYVYSRVKNLNSSRV
PDLLS.

Alternatively, in some embodiments, the envelope small membrane protein is the SARS-CoV-2 envelope small membrane protein with UniProtKB accession no. PODTC4, with the amino acid sequence set forth, below:

[SEQ ID NO: 5]
MYSFVSEETGTLIVNSVLLFLAFVVFLLVTLAILT
ALRLCAYCCNIVNVSLVKPSFYVYSRVKNLNSSRV
PDLLV.

Alternatively, the envelope small membrane protein may be a natural variant of the amino acid sequences presented above.

In some alternative embodiments, the plurality of antigens comprise a peptide with an amino acid sequence that corresponds to at least a portion of nucleoprotein and a peptide with an amino acid sequence that corresponds to at least a portion of membrane protein.

In some alternative embodiments, the plurality of antigens comprise a peptide with an amino acid sequence that corresponds to at least a portion of nucleoprotein and a peptide with an amino acid sequence that corresponds to at least a portion of envelope small membrane protein.

In some alternative embodiments, the plurality of antigens comprise a peptide with an amino acid sequence that corresponds to at least a portion of membrane protein and a peptide with an amino acid sequence that corresponds to at least a portion of envelope small membrane protein.

In some alternative embodiments, the plurality of antigens comprise a peptide with an amino acid sequence that corresponds to at least a portion of spike glycoprotein, a peptide with an amino acid sequence that corresponds to at least a portion of nucleoprotein, and a peptide that corresponds to at least a portion of membrane protein.

In some alternative embodiments, the at plurality of antigens comprise a peptide with an amino acid sequence that corresponds to at least a portion of spike glycoprotein, a peptide with an amino acid sequence that corresponds to at least a portion of nucleoprotein, and a peptide that corresponds to at least a portion of envelope small membrane protein.

In some alternative embodiments, the plurality of antigens comprises=a peptide with an amino acid sequence that corresponds to at least a portion of spike glycoprotein, a peptide that corresponds to membrane protein, and a peptide with an amino acid sequence that corresponds to at least a portion of envelope small membrane protein.

In some alternative embodiments, the plurality of antigens comprise a peptide with an amino acid sequence that corresponds to at least a portion of nucleoprotein, a peptide with an amino acid sequence that corresponds to at least a portion of membrane protein, and a peptide with an amino acid sequence that corresponds to at least a portion of envelope small membrane protein.

In some alternative embodiments, the plurality of antigens comprise a peptide with an amino acid sequence that corresponds to at least a portion of protein 3a, a peptide with an amino acid sequence that corresponds to at least a portion of non-structural protein 6, a peptide with an amino acid sequence that corresponds to at least a portion of ORF10 protein, and a peptide with an amino acid sequence that corresponds to at least a portion of ORF9B protein.

In some preferred embodiments, the protein 3a is the SARS-CoV-2 protein 3a as defined by UniProtKB accession no. PODTC3, with the amino acid sequence set forth below:

[SEQ ID NO: 6]
MDLFMRIFTIGTVTLKQGEIKDATPSDFVRATATI
PIQASLPFGWLIVGVALLAVFQSASKIITLKKRWQ
LALSKGVHFVCNLLLLFVTVYSHLLLVAAGLEAPF
LYLYALVYFLQSINFVRIIMRLWLCWKCRSKNPLL
YDANYFLCWHTNCYDYCIPYNSVTSSIVITSGDGT
TSPISEHDYQIGGYTEKWESGVKDCVVLHSYFTSD
YYQLYSTQLSTDTGVEHVTFFIYNKIVDEPEEHVQ
IHTIDGSSGVVNPVMEPIYDEPTTTTSVPL.

Alternatively, the protein 3a may be a natural variant of the amino acid sequence presented above.

In some preferred embodiments, the non-structural protein 6 is the SARS-CoV-2 non-structural protein 6 as defined by UniProtKB accession no. PODTC6, with the amino acid sequence set forth below:

[SEQ ID NO: 7]
MFHLVDFQVTIAEILLIIMRTFKVSIWNLDYIINL
IIKNLSKSLTENKYSQLDEEQPMEID.

Alternatively, the non-structural protein 6 may be a natural variant of the amino acid sequence presented above.

In some preferred embodiments, the ORF10 protein is the SARS-CoV-2 ORF10 protein as defined by UniProtKB accession no. A0A663DJA2, with the amino acid sequence set forth below

[SEQ ID NO: 8]
MGYINVFAFPFTIYSLLLCRMNSRNYIAQVDVVNFNLT.

Alternatively, the ORF10 protein may be a natural variant of the amino acid sequence presented above.

The ORF9B protein may be the SARS-CoV-2 ORF9B protein as defined by UniProtKB accession no. PODTD2, with the amino acid sequence set forth below:

[SEQ ID NO: 9]
MDPKISEMHPALRLVDPQIQLAVTRMENAVGRDQN
NVGPKVYPIILRLGSPLSLNMARKTLNSLEDKAFQ
LTPIAVQMTKLATTEELPDEFVVVTVK.

Alternatively, the ORF9B protein may be a natural variant of the amino acid sequence presented above.

In some alternative embodiments, the at least two coronavirus antigens comprises a peptide with an amino acid sequence that corresponds to at least a portion of non-structural protein 7a, a peptide with an amino acid sequence that corresponds to at least a portion of non-structural protein 7b, a peptide with an amino acid sequence that corresponds to at least a portion of non-structural protein 8, and a peptide that corresponds to at least a portion of uncharacterised protein 14.

The non-structural protein 7a protein may be the SARS-CoV-2 non-structural protein 7a protein as defined by UniProtKB accession no. PODTC7, with the amino acid sequence set forth below:

[SEQ ID NO: 10]
MKIILFLALITLATCELYHYQECVRGTTVLLKEPC
SSGTYEGNSPFHPLADNKFALTCFSTQFAFACPDG
VKHVYQLRARSVSPKLFIRQEEVQELYSPIFLIVA
AIVFITLCFTLKRKTE.

Alternatively, the non-structural protein 7a may be a natural variant of the amino acid sequence presented above.

The non-structural protein 7b protein may be the SARS-CoV-2 non-structural protein 7b protein as defined by UniProtKB accession no. PODTD8, with the amino acid sequence set forth below:

[SEQ ID NO: 11]
MIELSLIDFYLCFLAFLLFLVLIMLIIFWFSLELQ
DHNETCHA.

Alternatively, the non-structural protein 7b protein may be a natural variant of the amino acid sequence presented above.

The non-structural protein 8 protein may be the SARS-CoV-2 non-structural protein 8 protein as defined by UniProtKB accession no. PODTC8, with the amino acid sequence set forth below:

[SEQ ID NO: 12]
MKFLVFLGIITTVAAFHQECSLQSCTQHQPYVVDD
PCPIHFYSKWYIRVGARKSAPLIELCVDEAGSKSP
IQYIDIGNYTVSCLPFTINCQEPKLGSLVVRCSFY
EDFLEYHDVRVVLDFI.

Alternatively, the non-structural protein 8 may be a natural variant of the amino acid sequence presented above.

In some of the same embodiments and some other embodiments, the methods comprise: preparing an incubation composition by contacting a whole blood sample obtained from a human subject with a composition comprising a plurality of antigens corresponding to at least one coronavirus protein; and incubating the incubation composition for at least four hours; and measuring the presence or level of IFN-γ released due to the stimulation with the plurality of antigens; wherein the presence or quantity of detected IFN-γ is indicative of the level of cell-mediated immune responsiveness of the human subject.

In some embodiments of this type, the method further comprises measuring the presence or level of IL-2 released due to the stimulation with the plurality of antigens.

In some embodiments, the methods comprise: preparing an incubation composition by contacting a whole blood sample obtained from a human subject with a composition comprising a plurality of antigens corresponding to at least one coronavirus protein; and incubating the incubation composition for at least four hours; and measuring the presence or level of IL-2 released due to the stimulation with the plurality of antigens; wherein the presence or quantity of detected IL-2 is indicative of the level of cell-mediated immune responsiveness of the human subject.

In some embodiments of this type, the method further comprises measuring the presence or level of IFN-γ released due to the stimulation with the plurality of antigens.

In some embodiments, the plurality of antigens comprise a first peptide set that corresponds to a first coronavirus protein. Suitably, the first peptide set spans a region of the first coronavirus protein that is. In some preferred embodiments, the peptide set comprises peptides that overlap by one, two, three, four, five, six, seven, eight, nine, or ten or more amino acids. In some alternative embodiments, the peptide set comprises peptides that span the region of the first coronavirus protein, but do not overlap with one another.

In some embodiments, the plurality of antigens further comprises a second peptide set that corresponds to a second coronavirus protein. The second coronavirus protein may be a separate region or domain of the protein identified as the first coronavirus protein. Alternatively, the second coronavirus protein may be a different protein to that identified as the first coronavirus protein.

A composition comprising a biological sample from a subject, the sample comprising a plurality of antigens and a reagent for determining the level of an immune effector molecule.

In some embodiments, the immune effector molecule comprises a Th1 cytokine (e.g., one or both of IL-2 and IFN-γ). In some embodiments of this type the reagent is an antibody (e.g., a monoclonal antibody).

In some embodiments, the subject has previously been infected with a coronavirus (e.g., SARS-CoV-2).

In some of the same embodiments and some other embodiments, the biological sample is a whole blood sample. In some embodiments of this type, the composition further comprises an anticoagulant (e.g., heparin).

In yet another aspect, the present invention provides a method of determining the immunocompetency of a vaccinated subject to a virus protein, the method comprising contacting a sample from the subject with a plurality of peptide antigens corresponding to at least one virus protein, wherein the sample comprises immune cells capable of producing immune effector molecules following stimulation;

and detecting the presence or the level of an immune effector molecule in the sample; to thereby determine the immunocompetency of a vaccinated subject to a virus protein.

### BRIEF DESCRIPTION OF THE FIGURES

The following figures form part of the present specification and are included to further demonstrate certain aspects of the present disclosure. The disclosure may be better understood with reference to one or more of these figures in combination with the detailed description of specific embodiments presented herein.

FIG. 1 provides a schematic of the whole blood assay for the rapid detection of SARS-CoV-2-responsive T cell cytokine production. Whole blood (200 μL) was added into seven separate wells of a 96-well plate and stimulated under different conditions. Five wells were stimulated with CoV-peptide pools (CoV-1 to 5; from Table 1), the sixth condition was a positive control (mitogen) and the final well received no stimulation. The assay was incubated overnight for an average of 20 hours in a CO₂ controlled incubator. Following this, the plate was spun and plasma harvested for cytometric bead array analysis to detect production levels of 12 different cytokines.

FIG. 2 provides antigen-specific cytokine detection and principal component analysis demonstrating correlation between cytokine responses in recovered versus unexposed individuals. Box and whisker plots displaying cytokine responses to CoV pools 1-5 and control phytohemagglutinin (PHA) mitogen stimulation comparing COVID-19-recovered (red) and unexposed (blue) individuals. Statistical analysis confirmed significantly increased IFN-γ production (A) in recovered COVID-19 patients in response to CoV-3 (p=1.5e-5), CoV-4 (p=0.0054), CoV-5 (p=7.0e-5). Significantly increased IL-2 production (B) was observed in recovered COVID-19 patients in response to CoV-1 (p=0.0041), CoV-2 (p=0.036), CoV-3 (p=4.4e-09), CoV-4 (p=9.7e-09), CoV-5 (p=2.2e-07). Significantly increased IL-8 production (C) in recovered COVID-19 patients in response to CoV-1 (p=4.2e-07), CoV-2 (p=0.001), CoV-3 (p=1.1e-06) and CoV-4 (p=0.0047). Statistical analysis was performed using a Wilcoxon test. (*p =0.05, **p=0.005, ***p=0.0001). Principal component analysis of total cytokine production (D) in convalescent COVID-19 (n =44) patients (red) and unexposed (n=21) individuals (blue) across 12 cytokines (green). Whereby the direction and length of a green line indicates the degree of association between that cytokine and the surrounding patient cohort.

FIG. 3 provides a heat map of cytokine detection in whole blood following stimulation with SARS-CoV-2 peptide sets. Heat maps display cytokine response levels in Covid-19-recovered (Q) and unexposed (GR) individuals. Whole blood (200 μl) from recovered and unexposed individuals were incubated for 16 - 24 hours at 37° C. with peptide pools (CoV-1-CoV-5) containing overlapping peptide sets from SARS-CoV-2. Blood was incubated with a global mitogen stimulus (phytohemagglutinin) as a positive control, or left unstimulated/without peptide to control for spontaneously generated cytokine (background). Blood was then centrifuged and plasma harvested and analysed using a cytometric bead array to detect T-cell cytokines. Data represent the cytokine levels with background values subtracted from each of the peptide-stimulated culture conditions. Cytokine response levels were normalized within each individual cytokine and displayed using a colour (viridis) scale ranging from the lowest detection value of 0.0 (purple) to the highest detection value of 1.0 (yellow).

FIG. 4 provides a graphical representation of the association of IFN-γ and IL-2 in response to SARS-CoV-2 peptide pools. Data represent a correlation between IFN-γ and IL-2 production in response to (A) CoV-3 (r²=0.4162, p<0.001), (B) CoV-4 (r²=0.3078, p=0.002) and (C) CoV-5 (r²=0.6678, p<0.0001) peptide pools. Statistical analysis was performed to calculate the Pearson's correlation coefficient (r²) and statistical significance using a two-tailed t test (p).

FIG. 5 shows principal component analysis (PCA) demonstrating correlation between cytokine responses in recovered versus unexposed individuals. PCA of cytokine production in recovered Covid-19 patients (n=44) and unexposed (n=21) individuals. Data display the correlation between clustering of Covid-19 recovered (red) and unexposed (blue) individuals by detection of cytokines (A) IL-2 and (B) IFN-γ. (C) PCA for TNF production demonstrated no distinction between Covid-19 recovered (red) and unexposed (blue) cytokine responses.

FIG. 6 shows between sex, age and cytokine production in Covid-19 convalescent individuals. Comparison of the (A) IFN-γ and (B) IL-2 response to peptide pools CoV-1 to 5 and control phytohemagglutinin (PHA) in females and males. Data represents a comparison of the (C) IFN-γ and (D) IL-2 response to peptide pools CoV-1 to 5 and control PHA in people aged under 50 years and over 50 years. Statistical significance was determined using multiple t-tests in GraphPad Prism software, where the number of t-tests=6; (*p<0.05).

FIG. 7 provides a graphical representation of Immune cell phenotype in PBMC of recovered Covid-19 patients. PBMC were assessed for the presence of T cell, B cell and NK cell populations by flow cytometry. Data represents the median and range of cell populations as a proportion of total PBMC.

FIG. 8 provides graphical representations of expanded antigen-specific T-cell responses to SARS-CoV-2 proteins in COVID-19-convalescent individuals. Data represents CD4+IFN-γ⁺ (A) and CD8⁺IFN-γ⁺ (B) responses to individual SARS-CoV-2 proteins. (C) Polyfunctional analysis of dominant CD4⁺ responses to nucelocapsid (N), spike (S) and membrane (M) proteins. (D) Dominant CD8⁺ responses to N, S and ORF3a proteins.

FIG. 9 provides graphical representations of spike-specific cytokine detection following the first dose of ChAdOx1-S. Box and whisker plots displaying cytokine responses to (A) spike pool 1 and (B) spike pool 2 comparing vaccinated (PostVax; n=58 and unvaccinated individuals (PreVax, n=26). Data represent values after subtraction of background cytokine levels following incubation of blood with no antigen. Statistical analysis using multiple t tests (n=8) confirmed significant increases in the production of IFN-γ (P=0.0003), IL-2 (P=0.0011), IL-5 (P=0.0027), IL-10 (P=0.0037) and IL-13 (P=0.0001) in response to spike pool 1, and the production of IFN-γ (P=0.0004), IL-2 (P=0.0008), IL-5 (P=0.0017), IL-10 (P=0.0112), IL-17 (P=0.0049) and TNF (P=0.0155) in response to spike pool 2. Pairwise cytokine analysis from seven participants with PreVax and PostVax samples is shown in response to (C) spike pool 1 and (D) spike pool 2.

FIG. 10 provides graphical representations of proportional analysis of participants responding to either spike pool 1 or 2. (A) PostVax samples were considered cytokine positive if the response exceeded the maximum cytokine concentration from the PreVax cohort. Data represent the number of vaccinated participants generating a cytokine response to spike pool 1 and/or spike pool 2. (B) The heat map represents the pattern of cytokine responses from each vaccinated participant. Detected cytokines are represented by red boxes, while cytokines that were not detected are represented by grey boxes. (C) Data represents the concentration of IFN-γ and IL-2 from PostVax samples stimulated with either spike pool 1 or spike pool 2. Correlation analysis was performed using a Pearson correlation. (D) The heat map displays spike pool 1 and spike pool 2 aggregate cytokine response levels.

FIG. 11 provides graphical representations of the impact of sex, age and days post vaccination on cytokine responses. (A) Box and whisker plots displaying post-vaccination aggregate cytokine responses in females (n=41) and males (n=19). Data represent values after subtraction of background cytokine levels following incubation of blood with no antigen. Statistical significance was determined using multiple t-tests. No significant differences were observed. (B) Box and whisker plots displaying post-vaccination aggregate cytokine responses in participants <50 years of age (n=34) and >50 years of age (n=19). Data represent values after subtraction of background cytokine levels following incubation of blood with no antigen. Statistical significance was determined using multiple t-tests. (C—E) No significant differences were observed. Data represent correlation of the production of (C) IFN-γ, (D) IL-2, and (E) IL-13, with days post vaccination. No significant correlations were observed.

### DETAILED DESCRIPTION OF THE INVENTION

### 1. Definitions

Unless defined otherwise, all technical, and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e., to at least one) of the grammatical object of the article. By way of example, “a T cell” includes a single T cell, as well as two or more T cells; reference to “an antigen” includes a single antigen, as well as two or more antigens. Reference to “the disclosure” includes single or multiple aspects taught by the present disclosure; and so forth. All aspects of the invention are enabled within the width of the claims.

The term “about” is used herein to refer to conditions (e.g., amounts, concentrations, time, etc.) that vary by as much as 15%, 14%, 13%, 12%, 11%, 10%, 9%, 8%, 7%, 6%, 5%, 4%, 3%, 2%, or 1% to a specified condition.

By “antigen” is meant all, or part of, a protein, peptide, or other molecule or macromolecule capable of eliciting an immune response in a vertebrate animal, especially a mammal. Such antigens are also reactive with antibodies from animals immunized with that protein, peptide, or other molecule or macromolecule. The term includes any molecule or agent that can be bound by a major histocompatibility complex (MHC) or a non-classical MHC protein (e.g., CD1d or other CD1 family members).

The terms “patient”, “subject”, “host” or “individual” used interchangeably herein, refer to any subject, particularly a vertebrate subject, and even more particularly a mammalian subjection, for whom therapy or prophylaxis is desired. Suitable vertebrate animals that fall within the scope of the invention include, but are not restricted to, any member of the subphylum Chordata including primates, rodents (e.g., mice, rates, guinea pigs), lagomorphs (e.g., rabbits, hares), bovines (e.g., cattle), ovines (e.g., sheep), caprines (e.g., goats), porcines (e.g., pigs), equines (e.g., horses), canines (e.g., dogs), felines (e.g., cats), avians (e.g., chickens, turkeys, ducks, geese, companion birds), marine mammals (e.g., dolphins, whales), reptiles (e.g., snakes, frogs, lizards, etc.), and fish. A preferred subject is a human at risk of infection with a coronavirus.

As used herein, “stimulating” an immune or immunological response refers to administration of a composition that initiates, boosts, or maintains the capacity for the host's immune system to react to a target substance or antigen, such as a virus molecule, at a level higher than would otherwise occur. Stimulating a “primary” immune response refers herein to eliciting specific immune reactivity in a subject in which previous reactivity was not detected; for example, due to lack of exposure to the target antigen, refractoriness to the target, or immune suppression. Stimulating a “secondary” response refers to the re-initiation, boosting, or maintenance of reactivity in a subject in which previous reactivity was detected; for example, due to natural immunity, spontaneous immunisation, or treatment using one or several compositions or procedures.

### 2. Methods of Measuring Immune Response

According to a first aspect, a method is provided for measuring cell-mediated immune response activity, the method comprising contacting a sample comprising immune cells capable of producing immune effector molecules following stimulation with a plurality of peptide antigens corresponding to at least one coronavirus protein; and detecting the presence or the level of an immune effector molecule.

Advantageous embodiments and applications of said method are described herein. According to one embodiment of the first aspect, a method is provided for measuring cell-mediated immune response activity in a subject, the method comprising contacting a sample comprising immune cells capable of producing immune effector molecules following stimulation with a plurality of antigens, and detecting the presence or elevation in the level of an immune effector molecule.

The presence (or absence) or elevated level of the immune effector molecule is indicative of the level or capacity of cell-mediated immune responsiveness of the subject. In particular, said method allows to determine whether said subject has previously encountered the coronavirus antigen or an antigen for which the tested antigen is representative. Thereby, it can be determined whether the subject is capable of eliciting a cell-mediated immune response against the coronavirus antigen. In certain embodiments, also the quantitative level of cell-mediated immune responsiveness can be determined. The magnitude of the cell-mediated immune response detected in the assay presently disclosed can in certain embodiments be correlated to a disease state, progression and/or severity of a disease. Preferably, the magnitude of the cell-mediated immune response detected in the assay can be correlated to the risk of a subject of a subject being infected with the a coronavirus infection, by determining the subject's ability to generate an immune response to an virus antigen (i.e., the immunocompetence of the subject to the virus). Therefore, the present disclosure provides means to determine the cell-mediated immune responsiveness in a subject. The described method allows the determination of the level of immunocompetence and of immune cell responsiveness to a coronavirus. The assay also enables screening or monitoring of subjects previously exposed to a particular coronavirus, such as a viral envelope component.

The individual method steps and preferred embodiments of the method according to the first aspect of the invention will now be explained in detail.

A sample comprising immune cells capable of producing immune effector molecules following stimulation by an antigen is contacted with a plurality of peptide antigens corresponding to one or more coronavirus proteins. The immune cells and/or the whole sample can be obtained from a subject whose cell mediated immune responsiveness is to be determined.

Preferably, the subject is a human and the cell-mediated immune response method described herein is used in screening for responsiveness to viruses, and/or for determining the presence of any immunodeficiency or immunosuppression in a subject at risk of being infected by the virus. The latter may occur, for example, due to certain medicaments.

The sample comprises immune cells capable of producing immune effector molecules following stimulation with an appropriate antigen. “Immune cells” include but are not limited to lymphocytes including T cells, natural killer (NK) cells, B cells, macrophages and monocytes, dendritic cells or any other immune cell which is capable of producing one or more immune effector molecules in response to direct or indirect antigen stimulation. Preferably, the sample comprises lymphocytes, more preferably T lymphocytes. The terms “T cells” and “T lymphocytes” are used interchangeably herein. T cells are capable of eliciting a strong immune response if they recognize the offered antigen. If the T cells have been previously exposed to the tested antigen or an antigen for which the tested antigen is representative, a rapid re-stimulation of the T cells with specific memory of that antigen occurs. These antigen-specific T cells respond by secreting immune effector molecules (such as in particular IL-2). IL-2, or an immune effector molecule released in response to the released IL-2, can then be measured as specific marker of immune responsiveness against the tested antigen. In some embodiments, the sample comprises T lymphocytes, preferably CD4⁺ helper T cells and/or CD8⁺ cytotoxic T cells. In some embodiments, the sample is a body fluid comprising immune cells or is an immune cell-containing portion derived from a respective body fluid. In some preferred embodiments, the sample is whole blood. By “whole blood” is meant blood from a subject that has not been substantially diluted, altered, or fractionated. According to one embodiment the whole blood sample is peripheral blood. Notwithstanding that whole blood is the preferred and most convenient sample for determining cell-mediated immune response activity, also other samples containing immune cells can be used. Examples include but are not limited to lymph fluid, cerebral, fluid, tissue fluid (such as bone marrow or thymus fluid) and respiratory fluid including nasal and pulmonary fluid and bronchoalveolar lavage.

In some other embodiments, the sample may be a portion or derivative of the above-mentioned samples. For example, the sample may be depleted of cells unnecessary for measuring the cell mediated immune response, such as, whole blood being treated to remove components unnecessary for the cell-mediated immune response such as red blood cells and/or platelets. Suitable methods of fractionating/isolating cells in this way can be performed by methods well known in the art.

In some embodiments, the sample may be a peripheral blood mononuclear cells (PBMC) sample. PBMC samples can be obtained by methods well known in the art.

In some embodiments, the sample comprises cultured immune cells.

Furthermore, cryopreserved cells (e.g., cryopreserved PBMC cells), may be used as source of the immune cells of the subject. By way of an illustrative example of this type, thawed PBMC cells can be contacted with culture medium to provide the sample comprising immune cells which is then contacted and incubated with at least one coronavirus antigen. In some embodiments, the sample comprises all immune cells necessary for mediating a cellular immune response. However, it is also within the scope of the present invention to separately add stimulator cells, in particular antigen presenting cells (APC). In some embodiments, the sample comprises at least T cells (i.e., T lymphocytes). Optionally, the sample may also comprise NK cells (i.e., NK lymphocytes).

In some embodiments, the sample is not diluted by more than 50%, 40%, 30%, 25%, 20%, 15%, 10%, 5% or 3% prior to contacting the sample with the antigen at least one coronavirus.

The sample to be analysed is exposed to at least one coronavirus antigen.

In some embodiments, at least some of the plurality of antigens are immunogens. In some of the same embodiments and some other embodiments, at least some of the plurality of antigens are not immunogens. Antigens include (but are not limited to) peptides, proteins, haptens, allergens or toxins or any naturally occurring or synthetic molecule or parts thereof. In some embodiments, the antigen is selected from the group consisting of peptides, proteins (including glycoproteins), carbohydrates, phospholipids, phosphoproteins, phospholipoproteins, and fragments of the foregoing. The term “peptide” as used herein also includes polypeptides and proteins unless the context clearly indicates otherwise. The term “protein” also includes modified forms such as glycoproteins and phosphoproteins.

Typically, the plurality of antigens are peptides. Peptides used as antigens generally have a length selected from 5 to 100 amino acids, and preferably from 7 to 50 amino acids. Even more preferably, the peptides have a length of from 9 to 20 amino acids (i.e., 9, 10, 11, 12, 13, 14, 15, 16, 17, 18, 19 or 20 amino acids). In some embodiments, the plurality of antigens comprise a set of peptides from one or more different full length or part length proteins. A peptide set comprises at least two peptides and includes a series of overlapping or non-overlapping peptides. A respective set of peptides may cover the entire length of or a part of a naturally occurring protein antigen. However, the peptides do not necessarily have to be overlapping, or may overlap by a single amino acid or by multiple amino acids. In some preferred embodiments, the peptides overlap by between 1 and 10 amino acids (i.e., 1, 2, 3, 4, 5, 6, 7, 8, 9, or 10 amino acids).

In some preferred embodiments, the plurality of antigens comprise at least one peptide that is recognized by a CD8⁺ cytotoxic T cell. In embodiments of this type, the antigens are preferably peptides having a length of around 15 amino acids. Alternatively or in addition, the peptides may comprise 13 amino acids or less, 12 amino acids or less, 11 amino acids or less, or 10 amino acids or less. Suitable size ranges for a respective peptide that is recognized by a CD8⁺ cytotoxic T-cell include 7-14 amino acid residues, 7-13 amino acid residues, 8 to 12 amino acid residues, 8-11 amino acid residues, and 8 to 10 amino acid residues. Also, a set of peptides can be used which comprises, consists, or consists essentially, of peptides that at least some of which are recognized by CD8⁺ cytotoxic T-cells. Such peptides may encompass all or a part of a protein antigen such as a naturally occurring protein antigen.

The sets of peptides may cover the entire length of or a part of a protein antigen, e.g. a naturally occurring antigenic coronavirus protein. The peptides do not necessarily have to be overlapping or may overlap by a single amino acid or multiple amino acids. Such peptides may include a plurality of peptides which encompass and thus cover from 80-100% of an antigenic coronavirus protein. From “80-100%” means 80, 81, 82, 83, 84, 85, 86, 87, 88, 89, 90, 91, 92, 93, 94, 95, 96, 97, 98, 99 or 100%. Reference to a series of overlapping peptides from about 7 to 20 amino acid residues (e.g., 15 amino acid residues) in length which encompass all or part of a protein antigen according to one embodiment means a peptide of from about 7 amino acid residues in length to a maximum of 20 amino acid residues (e.g., 15 amino acid residues) which in total span from every amino acid residue which in total span amino acid residues to up to 6 amino acid residues of a protein antigen from its N-terminal end to its C-terminal end or part thereof. For example, if the length of a given peptide is x amino acid residues in length wherein x is from about 7 to 20, then the extent of overlap between two consecutive peptides is from x−1 to x−6. In some embodiments of this type, the overlap of each consecutive peptide is x−1.

The present disclosure includes the case where each peptide in the series or peptide set is the same length (i.e., x). However, the series of peptides or peptide set may comprise a mixture of x₁, x₂, xⱼ . . . xᵢ peptides where according to one embodiment each of xⱼ peptides is from about 7 to 14 amino acid residues in length or greater than 15 amino acid residues in length. The CD4⁺ and/or CD8⁺ peptides can be divided into separate pools of peptides. They can be added separately to the sample or can be included in one composition. This composition can then be exposed to the sample (for example for 16-24 hours at around 37° C.).

In some embodiments, one or more antigens are employed which mimic one or more of the effects of antigens presented to the immune system in vivo.

Typically, the plurality of peptides are derived from or cross-reactive with a protein antigen from a coronavirus.

By contacting the sample with the plurality of antigens, and optionally further additives, an incubation composition is provided. Preferably, said incubation composition is incubated above room temperature and thus at elevated temperatures. Preferably, the incubation temperature is above about 30° C., preferably above 35° C. Suitable ranges for the incubation temperature include 30° C. to about 38° C., preferably about 35° C. to about 38° C. Conveniently, the incubation composition is incubated at about 37° C. Preferably, the incubation composition is incubated for at least 4 hours at such elevated temperatures to allow stimulation of the immune cells by the plurality of antigens and the production of immune effector molecules. The incubation step may be from about 4 to about 50 hours, such as 4 to 40 hours, 5 to 30 hours, 8 to 24 hours, 16 to 24 hours, or a time period in between including 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 hours. In some embodiments, the incubation step may proceed for longer than 50 hours, for example for 3 days, 4 days, 5 days, 6 days, 7 days, 8 days, 9 days, or 10 days. In some embodiments, after an optional initial mixing step to distribute the antigens and the sample throughout the incubating composition, incubation is carried out without mixing further.

Optionally, one or more further additives can be added to the sample prior to, concurrently with, or sequentially to exposing the sample to the plurality of antigens. For example, one or more additives can be added that are necessary or advantageous for sample preparation and/or sample preservation such as a suitable anticoagulant if the sample is a blood sample (e.g., a whole blood sample). In some preferred embodiments, the anticoagulant is heparin. In some embodiments, of this type, the anticoagulant (e.g., heparin) is included in a blood collection tube prior to collecting the sample from the subject. Additives should not be comprised in a concentration wherein they could interfere with the cell-mediated immune response.

In some embodiments, the sample is contacted with a composition which comprises a plurality of antigens. Embodiments of this type are particularly advantageous, because the user does not have to add each antigen separately to the incubation composition. Providing such ready-to-use compositions avoids handling errors and saves hands on time. Optionally, a diluent or solvent is comprised in the composition comprising the plurality of antigens. Furthermore, one or more additives can be included in said composition if they are to be included in the incubation composition. The additives should not interfere with the cell-mediated response.

To prepare the incubation composition, the composition comprising the plurality of antigens, and optionally comprising one or more further additives such as an anticoagulant in case of a blood sample (e.g., a whole blood sample), is exposed to the sample. The sample can be added to the composition or vice versa. To prepare the incubation composition, the sample, the plurality of antigens, and the further additive (if present) are, preferably, mixed.

Examples of suitable composition forms include liquid compositions, semi-liquid compositions, gel-like composition and solid compositions, in particular dried compositions. According to some embodiments, the composition comprising the plurality of antigens, and optionally a further additive is comprised in a sample collection vessel, preferably a sample collection tube such as a blood collection tube. This is particularly convenient as the sample is directly contacted with the composition upon collection. According to one embodiment, the composition comprising the plurality of antigens, and optionally a further additive is spray-dried to the interior of the sample collection vessel. Suitable spray-drying methods are well-known in the prior art.

In some embodiments, the sample is obtained from a subject and is not diluted with any other composition, such as tissue culture medium, excipients or other liquid agents prior to contact with the at least one coronavirus antigen. In some typical embodiments, the incubation composition comprises at least 10% by volume sample. The term “at least 10% by volume” includes sample volumes of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, and 100% by volume of total incubation composition volume.

The methods of the invention also include the step of detecting the presence or elevation in the level of an immune effector molecule. As described above, the presence (which includes the absence) or level of an immune effector molecule is indicative of the level or capacity of cell-mediated immune responsiveness of the subject against the tested plurality of antigens. In particular, said method allows for a determination to be made as to whether said subject has previously encountered one or more of the tested plurality of antigens that correspond to a coronavirus or a coronavirus antigen that shows cross-reactivity with the coronavirus. Therefore, it can be determined whether the subject is capable of eliciting a cell-mediated immune response against said plurality of antigens, respectively the coronavirus that the tested antigens is representative for.

The detection of the immune effector molecule may occur at the peptide or protein level or at the nucleic acid level, in particular, the immune effector molecule mRNA expression level. Consequently, reference to detecting the “presence or level” of the immune effector molecule includes direct and indirect data. For example, the presence or amount of immune effector molecules can be directly determined using appropriate detection methods such as ELISA or ELISpot. However, in some embodiments, the presence or level of the immune effector molecule is measured based on its RNA expression level. High levels of immune effector molecule mRNA are indirect data showing increased levels of the immune effector molecule. Suitable methods for determining the mRNA expression level of a target gene are well-known in the prior art and therefore, do not need any detailed description. Accordingly, in some embodiments, the immune effector molecule may be detected using ligands or binding molecules such as antibodies specific for the effector molecule or by measuring the level of expression of genes encoding the immune effector molecule.

The immune effector molecules to be detected may be any of a range of molecules which are produced in response to cell activation, stimulation or re-stimulation by an antigen. Also, more than one immune effector molecule or a pattern of immune effector molecules released upon contact of the sample with the tested antigen can be detected. The immune effector molecule to be measured may be produced by immune cells, in particular can be produced by lymphocytes such as T cells, in particular CD4⁺ helper T cells and/or CD8⁺ cytotoxic T cells. Thus, in some embodiments, the method is based upon measuring the production of one or more immune effector molecules by cells of the immune system, in particular T cells, in response to antigenic stimulation. However, also non-immune cells may release immune effector molecules in response to the stimulation, or re-stimulation, of immune cells by the at least one coronavirus antigen as they are stimulated by the immune effector molecules that are released by the immune cells. For example, by immune effector molecules such as IL-2 and/or IFN-γ released by re-stimulated T-cells. These immune effector molecules can also be an important source of information. Therefore, according to an embodiment, the immune effector molecule in response to antigen re-stimulation. In other embodiments, a downstream immune effector molecule is measured. For example, IFN-γ or other immediate immune effector molecules produced by immune cells, in particular by T cells that are re-stimulated by the plurality of antigens. However, as described above, these molecules often induce or enhance the production of further immune effector molecules by other cells. The production of these further (downstream) immune effector molecules may also be measured. In some embodiments, the method further comprises detecting more than one type of immune effector molecule. For example, the presence or level of a pattern of immune effector molecules is detected either alone or in addition to immediate immune effector molecules such as IFN-γ. A respective pattern comprises more than two, preferably more than three different immune effector molecules. Analyzing a respective pattern can provide valuable information of the immune status of the subject. For example, specific immune effector molecules or patterns of immune effector molecules can be characteristic for a specific response to coronavirus infection.

The immune effector molecules to be measured are typically cytokines such as a lymphokine, interleukin, or chemokine. Preferably, the immune effector molecules include one or both of IFN-γ and IL-2. In some of the same embodiments and some other embodiments the immune effector molecules also include IL-8.

Other examples of suitable immune effector molecules include, but are not limited to, a range of cytokines such as interleukins (e.g., IL-4, IL-6, IL-8 (CXCL8), IL-10, IL-12, IL-13, IL-16 (LCF), IL-17, IL-1α (IL-1F1), IL-1β (IL-1F2), IL-1RA (IL-1F3), tumor necrosis factor (TNF), transforming growth factor beta (TGF-β), a colony stimulating factor (CSF) such as granulocyte-colony stimulating factor (G-CSF) or granulocyte macrophage-colony stimulating factor (GM-CSF), complement component 5a (C5a), GroA (CXCL1), sICAM-1 (CD54), IP-10 (CXCL10), I-TAC (CXCL11), MCP-1 (CCL2), MIF (GIF), MIP-la (CCL3), MIP-1β (CCL4), serpin El (PAI-1), RANTES (CCL5), or MIG (CXCL9). In some embodiments, the present invention provides methods wherein the immune effector molecule to be detected is a cytokine, a component of the complement system, perforin, defensin, cathelicidin, granzyme, Fas ligand, CD40 ligand, exotaxin, a cytotoxin, a chemokine, or a monokine.

According to a preferred embodiment, the present invention provides a method for measuring a cell-mediated immune response in a subject, said method comprising collecting a sample from said subject into a collection vessel wherein said sample comprises cells of the immune system which are capable of producing IFN-γ following stimulation by an antigen, incubating said sample with a plurality of antigens that correspond to one or more coronavirus proteins, and then measuring the presence of or elevation in the level of IFN-γ, whereby the presence or level of IFN-γ is indicative of the capacity of the subject to mount a cell-mediated immune response.

According to another preferred embodiment, the present invention provides a method for measuring a cell-mediated immune response in a subject, said method comprising collecting a sample from said subject into a collection vessel wherein said sample comprises cells of the immune system which are capable of producing IL-2 following stimulation by an antigen, incubating said sample with at least one coronavirus antigen, and then measuring the presence of or elevation in the level of IL-2, whereby the presence or level of IL-2 is indicative of the capacity of the subject to mount a cell-mediated immune response.

According to yet another preferred embodiments, the present invention provides a method for measuring a cell-mediated immune response in a subject, said method comprising collecting a sample from said subject into a collection vessel wherein said sample comprises cells of the immune system which are capable of producing IL-8 following stimulation by an antigen, incubating said sample with at least one coronavirus antigen, and then measuring the presence of or elevation in the level of IL-8, whereby the presence or level of IL-8 is indicative of the capacity of the subject to mount a cell-mediated immune response.

According to yet another preferred embodiment, the present invention provides a method for measuring a cell-mediated immune response in a subject, said method comprising collecting a sample from said subject into a collection vessel wherein said sample comprises cells of the immune system which are capable of producing IFN-γ and IL-2 following stimulation by an antigen, incubating said sample with a plurality of antigens that correspond to one or more coronavirus proteins, and then measuring the presence of or elevation in the level of IFN-γ and IL-2, whereby the presence or level of IFN-γ and IL-2 is indicative of the capacity of the subject to mount a cell-mediated immune response.

In some embodiments, method comprises collecting a sample from said subject into a collection vessel wherein said sample comprises cells of the immune system which are capable of producing IFN-γ and/or IL-2, following stimulation by an antigen, incubating said sample with a plurality of antigens that correspond to one or more coronavirus proteins, and then measuring the presence of or elevation in the level of IFN-γ and/or IL-2 in combination with IL-8, whereby the presence or level of IFN-γ and/or IL-2 and IL-8 is indicative of the capacity of the subject to mount a cell-mediated immune response.

Furthermore, the level of the one or more immune effector molecule may be screened alone or in combination with other biomarkers or disease indicators.

In some embodiments, the immune effector molecule is detected by using a ligand which specifically binds the immune effector molecule. Ligands to the immune effector molecules are particularly useful in detecting and/or quantitating these molecules. In some embodiments, the cells comprised in the sample are removed prior to detecting the immune effector molecule. Techniques for the detection assays that can be used are well known in the art and include, for example, radioimmunoassays, sandwich assays, ELISA and ELISpot. Antibodies to the immune effectors are particularly useful as ligands. Both polyclonal and monoclonal antibodies are obtainable by immunization with the immune effector molecules or antigenic fragments thereof and either type is utilizable for immunoassays. Methods of obtaining both types of antibodies are well known in the art. Polyclonal antibodies are less preferred but are relatively easily prepared by injection of a suitable laboratory animal with an effective amount of the immune effector, or antigenic part thereof, collecting serum from the animal and isolating specific sera by any of the known immunoadsorbent techniques. Although antibodies produced by this method are utilizable in virtually any type of immunoassay, they are generally less favored because of the potential heterogeneity of the product. The use of monoclonal antibodies in an immunoassay is particularly useful because of the ability to produce them in large quantities and the homogeneity of the product. The preparation of hybridoma cell lines for monoclonal antibody production derived by fusing an immortal cell line and lymphocytes sensitized against the immunogenic preparation can be done by techniques which are well known to those who are skilled in the art. Antibodies against specific immune effector molecules are also commercially available.

In some embodiments, detecting the presence or level of immune effector molecules comprises contacting the sample or a portion thereof (e.g., a PBMC fraction), with an antibody or a fragment thereof specific for the immune effector molecule to be detected for a time and under conditions sufficient for an antibody-effector complex to form, and then detecting said complex. As described above, cells comprised in the sample may be removed (for example, by centrifugation) prior to detection. By way of an illustrative example of this type, when using blood as sample, cells can be separated after incubation to the at least one coronavirus antigen.

A wide range of immunoassay techniques are available as can be seen by reference to U.S. Pat. Nos. 4,016,043; 4,424,279; and 4,018,653. Respective assays that can be used in conjunction with cell-mediated immune response tests described herein to detect the produced immune effector molecules are also described in International Patent Publication Nos. WO2004/042396, WO2008/113119, WO2010/009494 and WO2011/075773, herein incorporated by reference. Additionally, several methods for monitoring cellular immune responses are described in Clay et al., (2001), the methods taught therein also describing suitable assays for detecting the immune effector molecules produced in response to antigen re-stimulation. ELISA-based assays, ELISpot assays and nucleic acid-based assays such as the measurement of cytokine mRNA levels by real-time quantitative RT-PCR are described. Optionally, when determining the level of immune effector molecule based on its RNA expression level, the obtained data can be normalized to the expression of control gene, such as for example CD8. Respective methods can also be used in conjunction with the present invention to detect the produced immune effector molecules. In some embodiments, a nucleic acid-based assay for detecting the presence or level of an immune effector molecule is used. Nucleic acids, in particular RNA, can be isolated from the sample or the cellular portion thereof using standard methods well known in the prior art. Preferably, the presence or elevation of the expression of the immune effector molecule is detected in this embodiment using amplification-based assays, preferably PCR based assays.

Isolated RNA can first be reverse transcribed to cDNA prior to amplification using primers and/or probes specific for the immune effector molecule to be detected. Preferably, the detection is quantitative. One suitable method is quantitative real-time RT (reverse transcription) PCR.

Preferably, the detection of the immune effector molecule is a quantitative detection.

In some embodiments, the method of the invention further comprise comparing the detected immune effector molecule level or a value derived therefrom with a reference-level. This embodiment is particularly useful for diagnosing a coronavirus infection for which the antigen is representative. If said subject is infected with the coronavirus, the determined immune effector molecule level or a value derived therefrom is above the reference-level and if said subject is not infected with the pathogen, the determined immune effector molecule level or a value derived therefrom is below the reference-level. The same principle applies for determining if said subject is capable of mounting a cell-mediated immune response against a pathogen for which the antigen is representative.

In some of the same embodiments and some alternative embodiments, the sample is divided into at least two fractions. The first fraction of the sample is contacted with a plurality of antigens that correspond to one or more coronavirus proteins, to generate a response sample, and the second fraction of the sample is contacted with an inactive solution to generate a negative control sample. In embodiments of this type, the presence or level of the immune effector molecule is determined in the two fractions. Furthermore, the antigen-dependent immune effector molecule response of the sample is determined by subtracting the immune effector molecule level determined in the negative control sample from the immune effector molecule level determined in the response sample. The coronavirus antigen-dependent immune effector molecule response or a value derived therefrom is then compared with a reference-level, thereby providing an aid in determining whether the subject has previously encountered the antigen. It can therefore be determined whether the subject has generated immunological reactivity to the antigen, and/or whether the subject is at an increased susceptibility to an infection with a coronavirus.

Optionally, the method may further comprise dividing the sample into at least three fractions and incubating the third fraction of the sample with a T cell activator such as a mitogen to generate a positive control sample. Here, the immune cells may be incubated in three separate populations: (a) negative control sample (e.g., saline solution), (b) coronavirus antigen stimulated response sample; and (c) positive control sample (using for example, a T-cell activator). The immune effector molecule response of the positive control sample is determined by subtracting the immune effector molecule level determined in the negative control sample from the immune effector molecule level determined in the positive control sample and comparing the resulting immune effector response with a reference-level or a value derived therefrom.

The methods of the invention have particular utility in determining whether the subject has previously encountered any of the tested plurality of antigens or an antigen which shows cross-reactivity thereto and therefore generated immunological reactivity to the antigen. This provides a valuable aid in determining whether the subject has an active, a recent, or a latent infection, or if the subject is responding to treatment, is going to develop an infection, or is immunosuppressed. For example, if the coronavirus antigen-dependent immune effector molecule response is above the reference level, the result can be assessed as being positive, and a determination made that the subject is capable of eliciting a cell-mediated response against the coronavirus antigen. If the antigen-dependent immune effector molecule response is below the reference level and the positive control dependent immune effector response is above the reference level, the result can be assessed as being negative, and a determination made that the subject is not capable of eliciting a cell-mediated response against said antigen.

The mitogens that can be used in the present invention as positive control encompass all mitogens known by the skilled person and include but are not limited to phytohaemagglutinin (PHA), concanavalin A (conA) lipopolysaccharide (LPS) and pokeweed mitogen (PWM). Other examples of immune stimulants besides mitogens that can be used for providing a positive control include but are not limited to chemical compounds such as R848.

As described above, in some embodiments, the vessel in which the sample and at least one coronavirus plurality of antigens are co-incubated may also be the collection container used to collect the sample from the subject. Any one of a large number of different commercially available containers may be used, provided that they provide suitable sample dimensions. In some embodiments, the vessel is a tube which comprises a vacuum to facilitate the collection of the sample such as blood from a subject. Respective evacuated blood collection tubes are well-known in the prior art. In other embodiments, the vessel is a capillary tube. In some embodiments, a capillary tube is used to collect blood from the surface of the skin by capillary action. In some embodiments, the sample is collected from a subject into a collection vessel containing at least one coronavirus antigen, and at least one anti-coagulant (preferably, heparin), or to which the antigen, and an anti-coagulant (preferably, heparin), is subsequently added. In some embodiments, blood is sampled using a capillary sampling device such as a pin prick device and blood is collected into a heparinised collecting container and subsequently transferred into an appropriate container for co-incubation with the at least one coronavirus antigen. Preferably, the at least one coronavirus antigen, and optionally the anti-coagulant are provided in form of a single composition as described above. In some embodiments, whole blood from a subject is collected into a container containing the antigen, and optionally the anti-coagulant. In other embodiments, the antigen, and anticoagulant are added to the whole blood sample after collection.

Generally, the cells of the immune system lose the capacity to mount a cell mediated immune response in whole blood after extended periods following blood draw from the subject, and responses without intervention are often severely reduced or absent by 24 hours following blood draw. The reduction of labor and need for specialized equipment in the present invention allows cell mediated immune response stimulation with antigens to be performed at the point of care locations such as physicians' offices, clinics, and outpatient facilities. Once antigen stimulation is complete, the requirement for fresh and active cells no longer exists. IL-2, IFN-γ, IL-8 and other cytokines or immune effector molecules released due to the stimulation with the antigen are stable in cell-free or cell-depleted fluids such as plasma and, thus, the sample can be stored, or shipped without special conditions or rapid time requirements in a similar fashion to standard plasma or serum samples used for other infectious disease or other disease diagnosis. Therefore, a detection of the actual released immune effector molecules is preferred. However, the cells comprised in the incubation composition or the whole incubation composition can be contacted after incubation with a nucleic acid stabilizing composition which comprises reagents that stabilize the RNA expression pattern if the presence or level of immune effector molecule is determined based on the RNA expression level of the immune effector molecule. Several stabilizing compositions are commercially available. PreAnalytiX provides compositions that contain reagents for an immediate stabilisation of the RNA gene expression profile in blood. The respective compositions can also be used to stabilize the RNA gene expression profile of the cells comprised in the incubation composition. The respective stabilisation composition allows the transport and storage at room temperature without the risk of changes in the RNA profile by gene induction and transcript degradation (see for example U.S. Pat. Nos. 6,617,170, 7,270,953, Kruhoffer et al., 2007). The respective compositions are sold under the name of PAXgene Blood RNA Tubes.

Non-limiting applications of the methods of the invention, including uses, diseases and conditions will be described. The method according to the invention as well as the compositions and kits described herein can be widely used in the medical and diagnostic field. For example. The methods can be used in in vitro assays suitable for analysing the cell-mediated responsiveness of patients. Furthermore, the method according to the invention as well as the compositions and kits described herein are valuable analytical tools in order to test the immunocompetence of a subject at risk of a coronavirus infection.

That is, one aspect of the present application includes methods that demonstrate the cell-mediated immune responsiveness of a subject by measuring responsiveness to coronavirus antigen using the method according to the present invention. In some embodiments, a sample such as whole blood, an enriched white blood cell fraction or bronchoalveolar lavage may be obtained from a subject having or suspected of developing a particular disease (e.g., a disease caused by an infection with a coronavirus) and the immune responsiveness is measured by using the method according to the methods of the present invention described above and elsewhere herein; e.g., by detecting immune effector molecules released from effector T cells (e.g., CD4⁺ T cells and/or CD8⁺ cytotoxic T cells) in response to stimulation by the antigen.

According to one embodiment, the method is for determining if a subject is infected with and/or is capable of mounting a cell-mediated immune response against a coronavirus for which the antigen is representative. The method of the present invention can be used to serially monitor the level anti-coronavirus immunity, in persons at risk of coronavirus infection, as loss of this immune function may be associated with development of the disease such as a coronavirus disease. Preferably, the presence or level of one or both of IL-2 and IFN-γ is determined as the immune effector molecule in a respective test. A robust IL-2 and/or IFN-γ response induced by a coronavirus specific antigen indicates a decreased risk of the subject being infected with a coronavirus, as the patient has a strong cell mediated response and thus protection from the virus. A minimal or reduced IFN-γ response indicates an increased risk of the subject being infected with a coronavirus, as either no or a very low cell-mediated immunity exists. In some embodiments, the immune effector molecule tested in the methods described herein may also comprise IL-8.

According to one embodiment, the antigen is selected from the group consisting of peptides, proteins, including glycoproteins, phosphoproteins and phospholipoproteins, carbohydrates, phospholipids and fragments of the foregoing and preferably is provided by one or more peptides.

In some embodiments, two or more different antigens are used to stimulate the immune cells present in the sample, and/or two or more different effector molecules are detected.

### 3. Compositions, Kits and Uses

The present invention also provides a composition for inducing a cell mediated immune response in a sample, comprising
a) a sample comprising an immune cell;
b) at least one isolated SARS-CoV-2 antigen; and optionally,
c) at least one anticoagulant.

Details regarding the composition, the composition form, suitable antigens, and anticoagulants are as described above and elsewhere herein. Such composition can be used in the methods and applications described above. Preferably, the composition is a semi-liquid, gel-like or solid composition. Preferably, it is a dried composition. According to one embodiment, the composition is a spray-dried composition.

In some embodiments, the sample has one or more of the following characteristics:
i) the sample was obtained from a human subject;
ii) the sample was obtained from a human subject that is immunosuppressed or immunodeficient;
iii) the sample comprises immune cells selected from the group consisting of T cells, NK cells, B cells, dendritic cells, macrophages and monocytes; and/or
iv) the sample is whole blood.

In some of the same embodiments and some alternative embodiments, the at least one coronavirus antigen comprises is selected from the group consisting of peptides, proteins, including glycoproteins, phosphoproteins and phospholipoproteins, carbohydrates, phospholipids and fragments thereof. Preferably, the at least one coronavirus antigen comprises one or more peptides.

In some preferred embodiments, the composition comprises two or more different antigens.

In some embodiments, the composition comprises one or more peptides as antigen, wherein the one or more peptides have a length selected from 5 to 100 amino acids or 7 to 50 amino acids. In some particularly advantageous embodiments, the composition comprises as antigen one or more peptides that are recognized by a CD8⁺ cytotoxic T cell. Preferably, in this embodiment, the antigen is provided by one or more peptides having a length of less than 15 amino acids, preferably having a length selected from 7-14 amino acids. Details of the respective peptides were described above in conjunction with the method according to the first aspect and it is referred to the above disclosure.

Also provided is a sample collection vessel, in particular a sample collection tube (e.g., a blood collection tube), comprising a respective composition comprising:
a) at least one isolated antigen; and
b) at least one anticoagulant.

Preferably, the composition is sprayed onto the inside of the vessel which preferably is an evacuated blood collecting tube. By using such ready-to-use collection vessels in the method of the present invention, the conditions of the method are optimized and standardized and the handling is simplified.

Additionally, the present invention also provides a sample collection vessel, in particular a sample collection tube (e.g., a blood collection tube), comprising a composition comprising:
a) a blood sample;
b) at least one isolated SARS-CoV-2 antigen;
c) at least one anticoagulant.

The method according to the invention is preferably performed using a kit which provides the materials necessary for executing the method steps. Such a kit preferably comprises standardized material which ensures that the method is performed under optimized conditions, thereby ensuring that the results obtained from different samples or patients or by different practitioners are comparable to each other. Therefore, in a fourth aspect, the present disclosure also provides a kit for measuring cell-mediated immune response activity in a subject, comprising at least two antigens, at least one sample collection vessel, and at least one reagent for detecting at least one immune effector molecule. Preferably, the at least two coronavirus antigens are provided in form of a single composition. For this purpose, the composition according to the second aspect of the invention can be used and it is referred to the above disclosure for details of said composition. In some embodiments, the kit comprises a sample collection vessel such as a blood collection tube which comprises the composition comprising the at least one coronavirus antigen. Preferably, the composition additionally comprises an anti-coagulant such as heparin. As described, the composition comprised in the sample collection container can be the compositions described above and elsewhere herein. Preferably, the detection reagent is an immunodetection reagent such as a labeled antibody. However, for assays that are based on the detection of the mRNA expression level, the detection reagent can be provided by primers and/or probes specific for the immune effector molecule to be detected. Suitable assays and detection reagents are well known to the skilled person and were also described above.

According to a further aspect, the present invention provides a composition that comprises:
a) a sample comprising at least one immune cell;
b) at least one isolated coronavirus antigen;
c) at least one anticoagulant; and
d) at least one reagent for detecting at least one immune effector molecule.

This invention is not limited by the exemplary methods and materials disclosed herein. Numeric ranges described herein are inclusive of the numbers defining the range. The headings provided herein are not limitations of the various aspects or embodiments of this invention which can be read by reference to the specification as a whole. According to one embodiment, subject-matter described herein as comprising certain steps in the case of methods or as comprising certain ingredients in the case of e.g. compositions, solutions and/or mixtures refers to subject-matter consisting of the respective steps or ingredients. It is preferred to select and combine preferred embodiments described herein and the specific subject-matter arising from a respective combination of preferred embodiments also belongs to the present disclosure.

In order that the invention may be readily understood and put into practical effect, particular preferred embodiments will now be described by way of the following non-limiting examples.

### EXPERIMENTAL

### EXAMPLE 1

### Cellular Phenotyping of Blood from Study Subjects

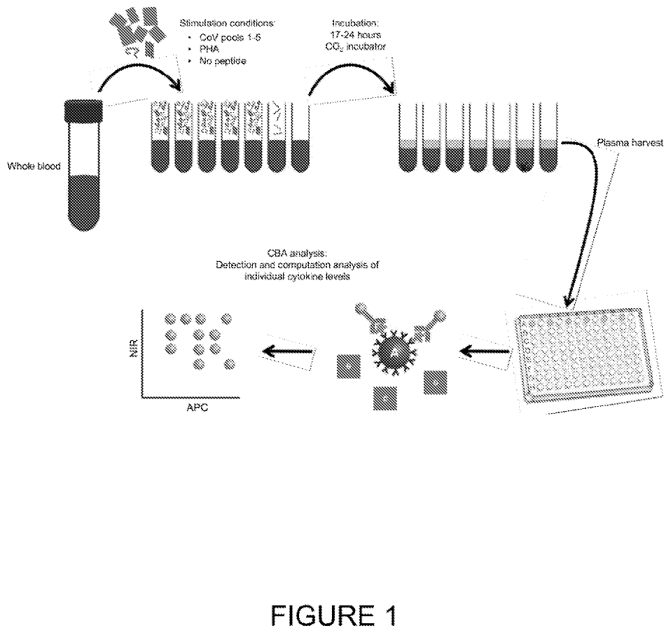

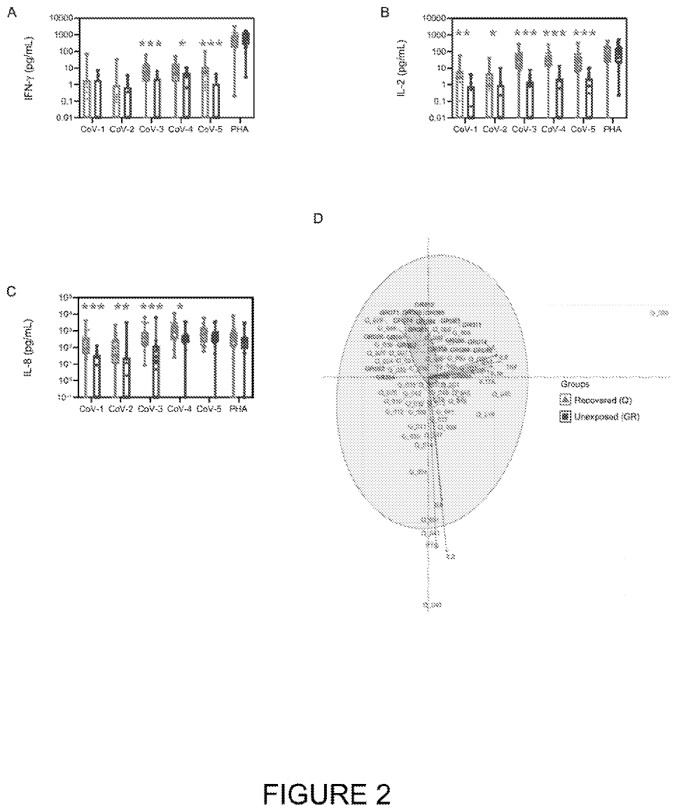

To determine whether antigen-specific T-cell immunity could be rapidly assessed in individuals who have recently recovered from Covid-19, a whole-blood assay was developed using overlapping peptide sets designed from SARS-CoV-2 antigens as stimuli (FIG. 1). Using this assay, whole blood from 44 Covid-19 convalescent patients were analysed, together with 21 healthy individuals that had been unexposed to the SARS-CoV-2 virus. Plasma harvested from the overnight stimulation was analysed by CBA, which identified significantly elevated levels of the T cell cytokines IFN-γ and IL-2 (FIG. 2A & B) in convalescent samples compared to unexposed samples. These responses indicate the successful generation of a T-helper 1 (Th1) immune response in convalescent individuals in response to SARS-CoV-2 peptide stimulation. In addition, convalescent patients displayed significantly increased levels of the pro-inflammatory cytokine IL-8 (FIG. 2C) compared to the unexposed cohort. Interestingly, minimal differences in the remaining cytokines (IL-1β, IL-4, IL-5, IL-6, IL-9, IL-10, IL-13, IL-17a and TNF) were observed between Covid-19-recovered individuals and unexposed individuals (data not shown). Principal component analysis (PCA) using the combined detection data from all analysed cytokines clearly stratified IL-2, IFN-γ, and IL-8 as the top cytokine candidates for differentiating between the SARS-CoV-2-specific responses of convalescent Covid-19 patients and unexposed individuals (FIG. 2D, shown in green). This PCA further demonstrates that the remaining cytokines analysed in this assay do not discriminate between convalescent and unexposed groups.

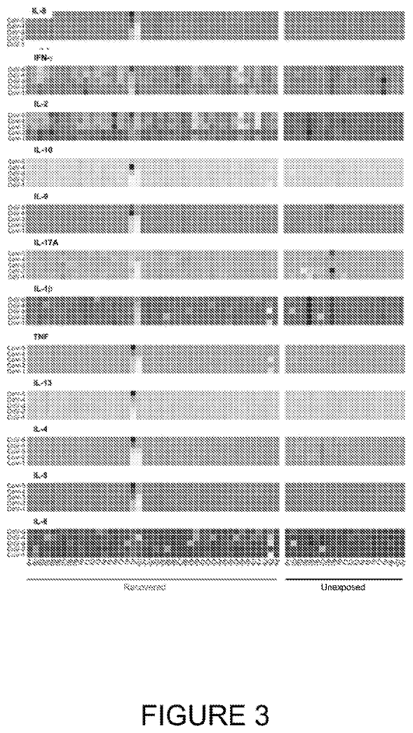

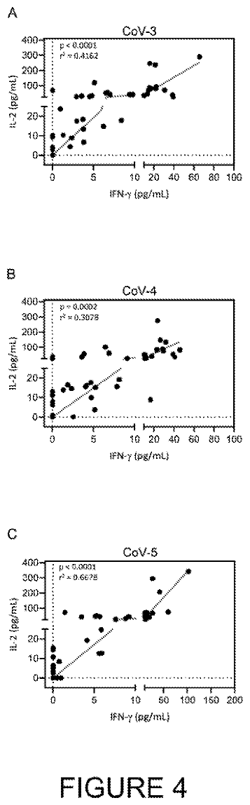

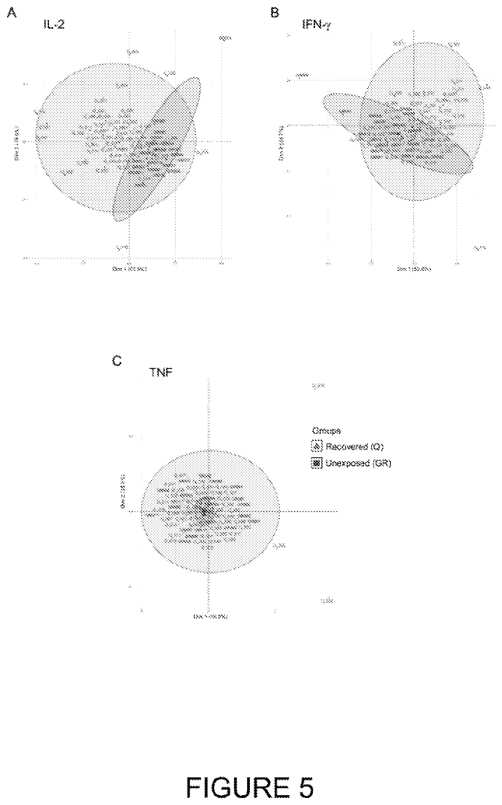

To illustrate the differential levels of cytokine detected from all blood samples (both convalescent and unexposed) in response to SARS-CoV-2 peptide stimulation, individual heat maps were generated (FIG. 3). Here, the total range of detection for each individual cytokine was normalized across all samples and displayed on a colour (viridis) scale ranging from 0.0 (purple) to 1.0 (yellow). This analysis distinguished elevated IFN-γ and IL-2 cytokine responses in convalescent patients compared to healthy donors when their blood was stimulated with SARS-CoV-2 peptide pools CoV-3, 4 and 5. Statistical analysis confirmed a significant correlation between the enhanced production of IL-2 and IFN-γ in convalescent patient responses to CoV-pools 3, 4 and 5 (FIG. 4). In comparison, the heat map illustrates that the remaining cytokines failed to differentiate between convalescent and unexposed individuals (FIG. 3). PCA completed on individual cytokine data sets confirmed that detection levels of IL-2 (FIG. 5A) and IFN-γ (FIG. 5B), could be used to group convalescent patients and unexposed individuals into two separate clusters. In contrast, PCA for TNF (FIG. 5C) failed to separate convalescent patient samples from unexposed samples.

### EXAMPLE 2

### Impact of Age on the Magnitude of Cytokine Response to SARS-CoV-2

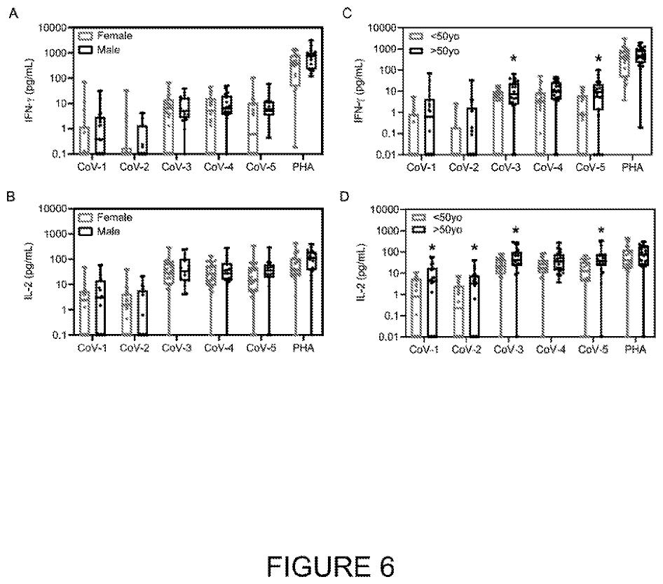

Given that a clear association between sex, age, and Covid-19 disease severity has been reported [12, 19], the inventors sought to determine the impact of these parameters on the magnitude of SARS-CoV-2 T cell responses in recovered individuals. No significant differences in IFN-γ or IL-2 responses to SARS-CoV-2 stimulation were observed between the sexes, suggesting that after the resolution of infection, both women and men generate a similar memory response (FIG. 6A, B). However, a clear difference in the magnitude of IFN-γ and IL-2 responses based on age was identified. Participants who were greater than 50 years of age at the time of recruitment generated a significantly higher IFN-γ response to the CoV-3 and 5 pools (FIG. 6C), and a significantly higher IL-2 response to the CoV-1, 2, 3 and 5 pools (FIG. 6D) than participants below 50 years of age.

### EXAMPLE 3

### SARS-CoV-2 Specific CD4⁺ and CD8⁺ Memory T Cell Immunity

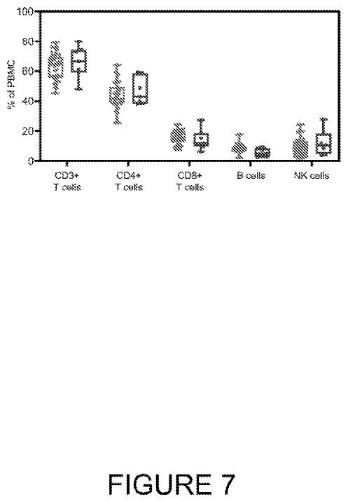

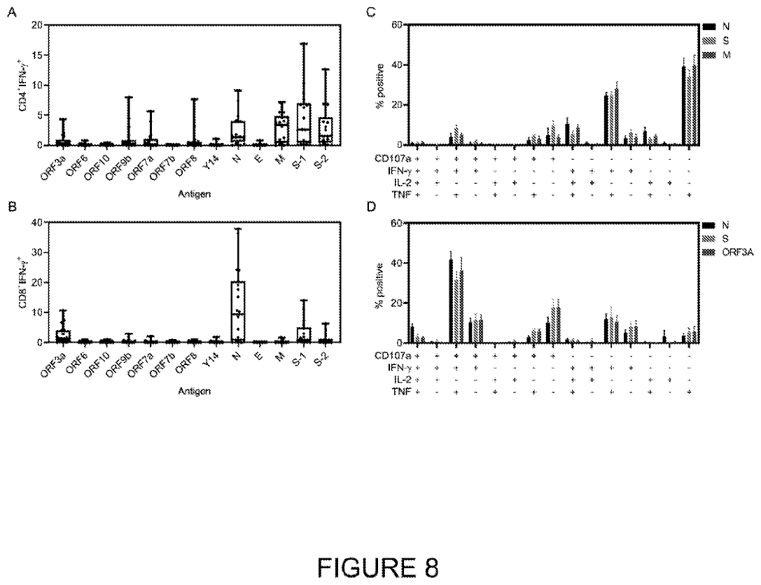

To determine if there were differences in the global immune composition of recovered individuals as compared to unexposed individuals, a phenotypic analysis on total PBMC was performed to compare lymphocyte populations between these cohorts (FIG. 7). Next, to validate the T cell reactivity detected in whole blood and to map the antigen-specificity of memory T cells, antigen-specific T cells were expanded from PBMC following stimulation with the SAR-CoV-2 peptide pools (Table 1). After 14 days in culture, cytokine production (IFN-γ, TNF, IL-2 and CD107a degranulation) was assessed following recall stimulation with each of the overlapping SARS-CoV-2 peptide pools. Strikingly, while a diverse IFN-γ response from CD4⁺ T cells was observed, which included recognition of the proteins spike (S), nucleocapsid (N) and membrane (M) together with ORFs 3a, 7a, 8 and 9b (FIG. 8A), the predominant memory CD8⁺ T cell response was directed against the N protein (FIG. 8B). Of the 18 recovered individuals assessed, a majority showed expansion of N protein-specific T cells, with a median frequency of 9.36%, whereas the median response to ORF3a was 1.36%. Additionally, a consistent low-level recognition of the two spike pools was observed. Polyfunctional analysis on expanded T cells revealed a similar response profile to each dominant antigen, with CD4⁺ T cells recognizing N, S or M characterized by the single production of TNF or co-expression of TNF and IFN-γ (FIG. 8C). CD8⁺ T cells predominantly produced TNF and IFN-γ, with a high proportion also demonstrating degranulation (CD107a). In contrast to the ex vivo whole-blood analysis little IL-2 production was observed. This suggests that these memory T cells undergo functional changes following in vitro proliferation and differentiation.

TABLE 1

| | |
|---|---|
| CoV-1 | Protein 3a; Non-structural protein 6; ORF10 protein; |
| | ORF9B protein |
| CoV-2 | Non-structural protein 7a; Non-structural protein 7b; |
| | Non-structural protein 8; Uncharacterised protein 14 |
| CoV-3 | Nucleoprotein; membrane protein; envelope small |
| | membrane protein |
| CoV-4 | Spike glycoprotein C-terminus region |
| CoV-5 | Spike glycoprotein N-terminus region |
| SARS-CoV-2 | Nucleoprotein; Membrane protein; Envelope small |
| | membrane protein; Spike glycoprotein |

### Discussion

T cells play an essential role in the control of human viral infections. In the absence of T cell immunity, chronic infection occurs which typically requires ongoing anti-viral treatment. Similarly, early observations suggest that the induction of a SARS-CoV-2 specific T cell response is likely important in the control of Covid-19, whereby severe disease is associated with lymphopenia and checkpoint marker expression on T cells [11, 12, 18]. The capacity to rapidly detect the induction of antigen-specific immunity following diagnosis could provide a means of stratifying patient risk, particularly with regard to the onset of severe disease, and potentially assist in identifying individuals who require earlier intervention with anti-viral therapy and other treatment. These data demonstrate a simple whole-blood assay that can rapidly detect cytokine production in response to SARS-CoV-2 antigens. In recovered people, the assay confirmed a predominantly Th1-cytokine response driven by antigen-specific T cells, and identified by detection of IFN-γ and IL-2 in response to both structural and non-structural SARS-CoV-2 proteins.

The results from this study confirm recent observations demonstrating a bias towards T cell responses against SARS-CoV-2 structural antigens in recently recovered patients [14, 20]. Whilst further work is required to delineate and define the key T cell antigenic determinants in SARS-CoV-2, it is clearly evident in this cohort that most people who successfully resolve infection generate readily detectable T cell immunity, a majority of which is raised against the N protein. This finding is supported by other recent studies on SARS-CoV-2 T cell immunity [14, 15, 20-22]. Based on observations in other coronavirus infections, particularly in animal models, it is also likely that this T cell response plays a critical role in disease prevention. Ongoing studies in patients with acute infection and symptomatic disease should help to further define the role of T cells in the prevention of Covid-19. Longitudinal analysis may be necessary to provide further insight into the stability of these T cell responses over time.

Despite the known importance of T cells in the control of most human viruses, very few clinically approved diagnostic tests are available to study T-cell immunity, with the exception of the QuantiFERON-CMV whole-blood assay, currently employed to assess cytomegalovirus (CMV) infection in solid organ transplant patients. In this context, the QuantiFERON-CMV assay is clinically approved to assess CMV-specific T-cell immunity, and is used successfully to stratify patient risk [23]. This knowledge has allowed for improved implementation of anti-viral therapies. The results of our current study demonstrate that a similar rapid whole-blood test can effectively assess SARS-CoV-2-specific T cell immune response.

The ease of application of this SARS-CoV-2 whole-blood assay will provide an effective means to study SARS-CoV-2-specific T cell immunity in large numbers of patients, particularly during the acute stages of infection. We anticipate that this approach will also provide a rapid and early clinical risk assessment for patients that have failed to generate effective immunity, and therefore require additional therapeutic intervention, such as anti-viral or immunotherapeutic treatment.

### Materials & Methods

Study subjects

This study was performed according to the principles of the Declaration of Helsinki. Ethics approval to undertake the research was obtained QIMR Berghofer Medical Research Institute Human Research Ethics Committee. Informed consent was obtained from all the participating patients. The inclusion criteria for the study was that volunteers had been diagnosed with SARS-CoV-2 infection and had subsequently been released from isolation. A total of 30 volunteers were recruited in May and June 2020. Volunteer demographics are shown in Table 1. Blood samples were collected from all volunteers to assess whole blood SARS-CoV-2 T-cell immunity, and peripheral blood mononuclear cells (PBMC) were isolated to assess cellular phenotype.

Whole Blood Measurement of SARS-CoV-2 Specific T Cell Immunity

A small-scale whole blood assay was initially used to recall cytokine production in response to SARS-CoV-2. Heparinised whole blood (200 μl) was aliquot into ten wells of a 96-well V-bottom plate. To six wells, 1 μg/ml per peptide of SARS-CoV-2 overlapping peptide pools were added as outlined in Table 1. SARS-CoV-2 overlapping peptide pools were supplied by JPT Technologies. Wells containing no peptide, the mitogen phytohemagglutinin (PHA), a custom Epstein Barr Virus (EBV) peptide pool and a custom cytomegalovirus (CMV) peptide pool were used as controls. The plate was incubated for 16 to 24 hours at 37° C. and plasma supernatant collected and stored at -80° C. before use.

T Cell Cytokine Analysis

A cytometric bead array (CBA) was used to quantify the production of the T-cell cytokines IL-2, IL-4, IL-5, IL-9, IL-10, IL-13, TNF and IFN-γ. The CBA was performed using the BD Biosciences Flex-sets. Samples were acquired using a BD LSRFortessa with FACSDiva software (BD Biosciences). Post-acquisition cytokine analysis was performed using FCAP array (BD Biosciences) software.

PBMC Immunophenotyping

Immune cell phenotyping to assess the frequency and absolute number of T cell subsets, B cell subsets, NK cell subsets and monocytes was performed on peripheral blood mononuclear cells (PBMC) isolated from all volunteers. In a BD Trucount tube, 50 μl of PBMC were incubated with the following antibodies, anti-CD3 BV711, anti-CD4 AF700, anti-CD8 SB780, anti-CD16 PE, anti-CD19 PE-Cy5, anti-CD27 PE-Dazzle594, anti-CD28 BV480, anti-CD38 PERCP-Cy5.5, anti-CD45 APC, anti-CD45RA FITC, anti-CD56 BV421, anti-CCR7 PE-Cy7 and live/dead fixable near-IR (Life Technologies). Following incubation, cells were directly fixed with paraformaldehyde and acquired using a BD LSRFortessa with FACSDiva software (BD Biosciences). Post-acquisition analysis was performed using FlowJo Version 10 software (FlowJo LLC, Ashland, Oregon, USA).

Statistical Analysis

GraphPad Prism 8.2.1 (San Diego, CA, USA) was used to perform statistical analysis. Demographics and clinical characteristics of patients were represented as median (range) and n(%), respectively. Statistical comparisons were made using nonparametric unpaired t test (Mann-Whitney U test).

Correlative analysis was performed using a nonparametric spearman correlation. Graphs were prepared in GraphPad Prism. Box plots were used to represent median (horizontal line), 25th and 75th percentiles (boxes) and 5th and 95th percentiles (whiskers). P<0.05 was considered statistically significant.

### EXAMPLE 4

### Immune Response in Vaccinated Individuals

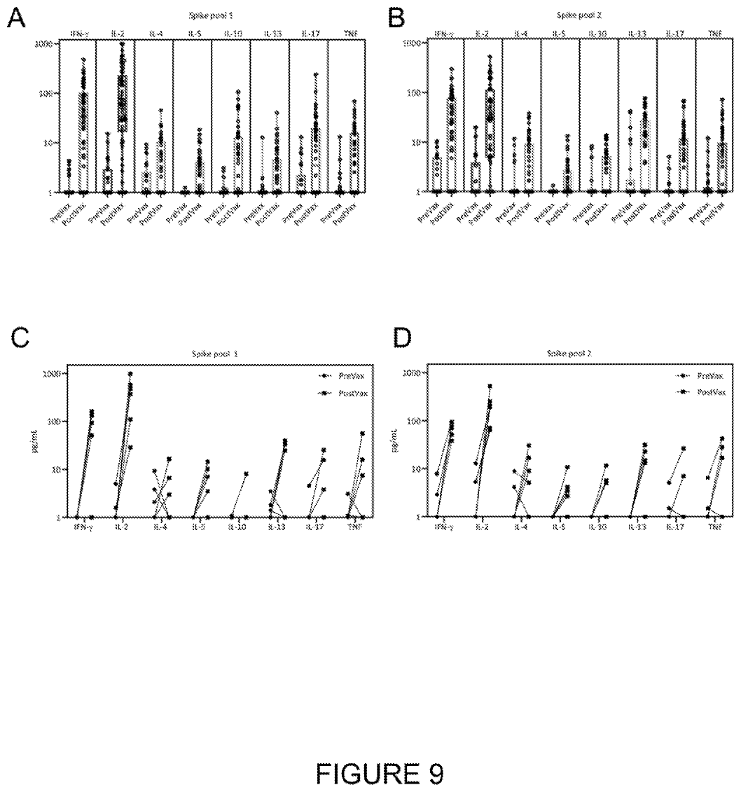

To determine if a similar approach could be used for the high-throughput screening of vaccine recipients, inventors recruited a cohort of 58 recipients of the ChAdOx1-S)(AstraZeneca® Covid-19 vaccine 11-23 days after their first dose (PostVax). To assess vaccine-induced T cell responses, peripheral blood was incubated overnight with two separate pools of overlapping peptides covering the SARS-CoV-2 spike antigen. Plasma was harvested and cytokine production was determined using similar CBA to that described above. To accommodate the potential impact of pre-existing or cross-reactive immune responses in the detection of spike-specific immunity, a cohort of 26 non-vaccinated and SARS-CoV-2 unexposed donors were used as a control (PreVax). Similar to the observations in SARS-CoV-2-recovered individuals, a significant increase in the quantity of the two Th1 cytokines (i.e., IFN-γ and IL-2) produced was observed in response to both spike peptide pools in the PostVax cohort (FIG. 9A, B). The inventors also noted a significant increase in the production of all other cytokines measured, excluding IL-4, in response to at least one of the peptide pools following vaccination. Pair-wise analysis from five participants for whom we had both PreVax and PostVax samples confirmed an increase in cytokine production in 4/5 participants (FIG. 9C, D). However, the inventors also noted that the increase in cytokine production was not evident in all vaccine recipients, with some demonstrating no detectable change in their cytokine response.

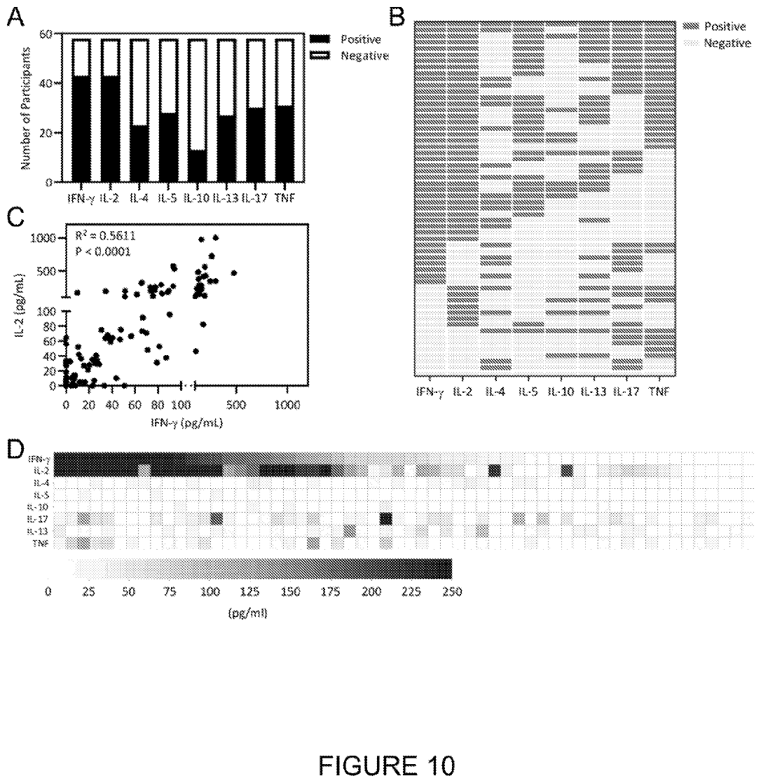

To set a stringent cut-off for the detection of a cytokine response, the maximum value detected in the PreVax cohort was used to determine a baseline for each cytokine. As such, PostVax participants were only considered positive if their cytokine response was greater than the established PreVax cut-off. The data was collated from both peptide pools to determine the number of participants with a positive cytokine response (FIG. 10A). As anticipated, IFN-γ and IL-2 responses were the most frequently produced cytokines, detected in samples from 43 participants. Assessment of these cytokine profiles revealed that IFN-γ and IL-2 were produced by T cells from people with strong SARS-CoV-2 T-cell immunity, while the production of other cytokines was variable (FIG. 10B). T cells from 51/58 (87.9%) individuals produced either IFN-γ or IL-2, and both cytokines were detected in 37/58 (63.8%) of these samples. Of the remaining seven (12.1%) participants whose T cells did not express detectable IFN-γ or IL-2 following spike peptide stimulation, six showed detectable responses to other cytokines (IL-4, IL-5, IL-10, IL-13, IL-17 or TNF), while one had no detectable cytokine responses after the first vaccine dose. To determine if there was a correlation between the quantities of different cytokines, we compared the quantity of IFN-γ with all other cytokines. Consistent with published analysis in SARS-CoV-2-recovered individuals, a strong correlation was evident between IFN-γ and IL-2 (FIG. 10C). The present inventors also noted a weak correlation between IFN-γ and IL-5, and IFN-γ and TNF (data not shown). No correlation was evident between IFN-γ and other cytokines. The heat map in FIG. 10D further emphasises the strong association between IFN-γ and IL-2 production, and no correlation between the levels of other cytokines.

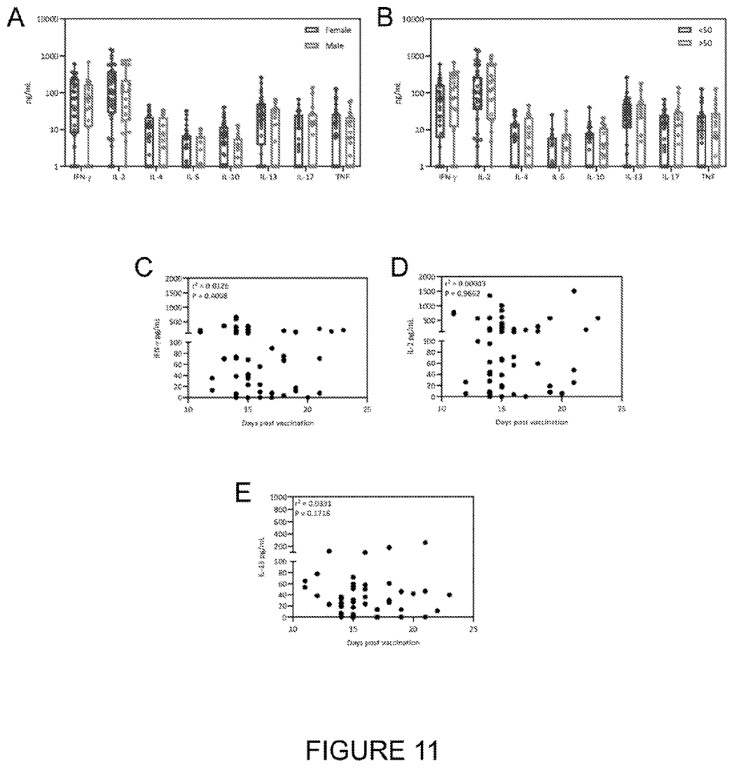

Age and sex have both been shown to impact risk of Covid-19 [19]. Age is also well known to have an impact on vaccine responsiveness [24]. To assess the impact of these factors on T-cell responses following the first dose of the ChAdOx1-S vaccine, the inventors compared cumulative spike-specific cytokine responses in females and males, and in younger (<50 years of age) and older vaccine recipients (>50 years of age) from 53 participants for whom we had year of birth available. No significant differences in the cytokine responses in females and males were noted, nor in younger or older vaccine recipients (FIG. 11A, B). Participants who demonstrated no detectable IFN-γ and/or IL-2 response following the first dose were seen in all sub-groups. Although only one donor was aged greater than 70, their T cells generated both IFN-γ and IL-2 post vaccination. To assess the impact of time post vaccination on cytokine titres, we correlated cytokine responses with days post vaccination. There was no correlation between days post vaccination and titres of IFN-γ (FIG. 11C), IL-2 (FIG. 11D) or IL-13 (FIG. 11E). High cytokine responses were evident within 11 days and up to 23 days following vaccination. These observations indicate that differences in the magnitude of the immune response against SARS-CoV-2 are attributable to factors independent of sex, age and time post vaccination in this cohort.

### Discussion

The worldwide implementation of a number of different Covid-19 vaccines has provided hope that the Covid-19 pandemic will soon be under control [25-27]. However, the emergence of multiple SARS-CoV-2 variants that may limit the effectiveness of vaccine-induced neutralising antibodies [28, 29], highlights the need to understand all aspects of vaccine induced immunity. In this study, the present inventors demonstrate that the majority of ChAdOx1-S vaccinees generate a strong SARS-CoV-2 spike-specific T cell response within 2-3 weeks of their first dose. However, we also noted that a Th1 cytokine (IFN-γ and/or IL-2) response was not detectable in 7 of 58 (12.1%) participants. It remains to be determined if the response in these individuals will be boosted following administration of the second dose or if they represent a proportion of the population who do not generate significant T cell immunity against the spike antigen.

The observations in our current study are consistent with those reported in the analysis of T cell responses in a phase 1/2 study of the ChAdOx1-S vaccine [30]. Similarly, an increase in the spike-specific production of IFN-γ and IL-2 by PBMC was evident in vaccine recipients after a single dose. It was also evident in this study that some recipients did not generate responses above those detected in non-vaccinated individuals. While the threshold for effective T cell immunity has not been established, these observations suggest that some individuals may not generate efficient T cell responses following a single dose. Observations from the initial phase 1/2 study indicate that these low responses may be boosted following the second dose [31]. Most recently, studies have also suggested that these responses could be further amplified using a heterologous boost with the BNT162b2 (PFIZER® vaccines [32]. In this study, the ChAdOx1-S vaccine did not appear to significantly augment spike-specific T cell immunity.

To provide primary assessment of parameters that may impact the T cell response generated post vaccination, we assessed the magnitude of the response in the context of sex and age. As previously reported, the participant's sex did not impact the magnitude of the T cell response [30]. These data also demonstrate no impact of age on the magnitude of the T-cell response, although this cohort was primarily limited to participants between the ages of 27 and 61, with only a single participant over the age of 70. However, given the variability in responsiveness identified in our cohort, it is likely that additional drivers of differential T cell immunity post vaccination are involved, including co-morbidities (which we did not collect in this cohort). Another potentially influential variable lays with patient-specific human leukocyte antigens, which is known to influence vaccine responsiveness [33, 34]. It also remains to be determined if differential T-cell immunity correlates with antibody-mediated immunity.

Taken together, these data evidence that whole-blood cytokine assays can provide an effective and rapid strategy for the high-throughput assessment of T cell immunity following vaccination for Covid-19, providing a platform for expansion into larger cohorts, particularly those known to be at an increased risk of developing severe Covid-19.

### Materials & Methods

Study participants

This study was performed according to the principles of the Declaration of Helsinki. Ethics approval to undertake the research was obtained from QIMR Berghofer Medical Research Institute Human Research Ethics Committee. Informed consent was obtained from all participants. Peripheral blood samples were collected from 58 participants who had received the first dose of vaccine between 11 and 23 days previously. A cohort of 26 healthy, non-vaccinated and non-Covid-exposed individuals was also recruited to determine positive cytokine responses.

Whole-Blood Measurement of SARS-CoV-2-Specific T-Cell Immunity

The small-scale whole-blood assay used to assess T cell responses following vaccination has recently been described. The protocol was modified to only assess spike-specific T cell immunity. The peptide pools using in the study were supplied by JPT Technologies (Berlin, Germany). A T cell cytometric bead array (CBA) was performed using the BD Biosciences Flex-sets (Franklin Lakes, New Jersey). Samples were acquired using a BD LSRFortessa with FACSDiva software (BD Biosciences) and analysed using FCAP array (BD Biosciences) software.

Statistical Analysis

GraphPad Prism 8.2.1 (San Diego, CA) was used to perform statistical analysis. Statistical comparisons between participant groups (post-Vax and unvaccinated) were made using multiple unpaired t tests using the Holm-Sidak Method. Correlative analysis was performed using the Pearson correlation co-efficient. Box plots were used to represent median (horizontal line), 25^{th} and 75^{th} percentiles (boxes) and minimum and maximum values (whiskers). P<0.05 was considered statistically significant.

### BIBLIOGRAPHY

1. (WHO), W. H. O. WHO Coronavirus Disease (COVID-19) Dashboard.

2. Amanat, F. & Krammer, F. SARS-CoV-2 Vaccines: Status Report. Immunity 52, 583-589, doi:10.1016/j.immuni.2020.03.007 (2020).

3. Sharpe, H. R. et al. The early landscape of COVID-19 vaccine development in the UK and rest of the world. Immunology, doi:10.1111/imm.13222 (2020).

4. Thanh Le, T. et al. The COVID-19 vaccine development landscape. Nat Rev Drug Discov 19, 305-306, doi:10.1038/d41573-020-00073-5 (2020).

5. Mueller, S. N. & Mackay, L. K. Tissue-resident memory T cells: local specialists in immune defence. Nat Rev Immunol 16, 79-89, doi:10.1038/nri.2015.3 (2016).

6. Dunn, G. P., Bruce, A. T., Ikeda, H., Old, L. J. & Schreiber, R. D. Cancer immunoediting: from immunosurveillance to tumor escape. Nat Immunol 3, 991-998, doi:10.1038/ni1102-991 (2002).

7. Sprent, Surh, C. D. T cell memory. Annu Rev Immunol 20, 551-579, doi:10.1146/annurev.immnunol.20.100101.151926 (2002).

8. Welsh, R. M. & Selin, L. K. No one is naive: the significance of heterologous T-cell immunity. Nat Rev Immunol 2, 417-426, doi:10.1038/nri820 (2002).

9. Wherry, E. 3. & Ahmed, R. Memory CD8 T-cell differentiation during viral infection. 3 Virol 78, 5535-5545, doi:10.1128/JVI.78.11.5535-5545.2004 (2004).

10. Wherry, E. 3., Blattman, 3. N., Murali-Krishna, K., van der Most, R. & Ahmed, R. Viral persistence alters CD8 T-cell immunodominance and tissue distribution and results in distinct stages of functional impairment. J Virol 77, 4911-4927, doi:10.1128/jvi.77.8.4911-4927.2003 (2003).

11. Diao, B. et al. Reduction and Functional Exhaustion of T Cells in Patients With Coronavirus Disease 2019 (COVID-19). Front Immunol 11, 827, doi:10.3389/fimmu.2020.00827 (2020).

12. Zhou, F. et al. Clinical course and risk factors for mortality of adult inpatients with COVID-19 in Wuhan, China: a retrospective cohort study. Lancet 395, 1054-1062, doi:10.1016/S0140-6736(20)30566-3 (2020).

13. Giamarellos-Bourboulis, E. 3. et al. Complex Immune Dysregulation in COVID-19 Patients with Severe Respiratory Failure. Cell Host Microbe, doi:10.1016/j.chom.2020.04.009 (2020).

14. Grifoni, A. et al. Targets of T Cell Responses to SARS-CoV-2 Coronavirus in Humans with COVID-19 Disease and Unexposed Individuals. Cell, doi:10.1016/j.ce11.2020.05.015 (2020).

15. Takuya Sekine, A. P.-P., et al., Robust T cell immunity in convalescent individuals with asymptomatic or mild COVID-19. Cell.

16. Chour, W. et al. Shared Antigen-specific CD8+T cell Responses Against the SARS-COV-2 Spike Protein in HLA A*02:01 COVID-19 Participants. medRxiv, 2020.2005.2004.20085779, doi:10.1101/2020.05.04.20085779 (2020).

17. Weiskopf, D. et al. Phenotype and kinetics of SARS-CoV-2-specific T cells in COVID-19 patients with acute respiratory distress syndrome. Sci Immunol 5, doi:10.1126/sciimmunol.abd2071 (2020).

18. Zheng, H. Y. et al. Elevated exhaustion levels and reduced functional diversity of T cells in peripheral blood may predict severe progression in COVID-19 patients. Cell Mol Immunol, doi:10.1038/s41423-020-0401-3 (2020).

19. Gebhard, C., Regitz-Zagrosek, V., Neuhauser, H. K., Morgan, R. & Klein, S. L. Impact of sex and gender on COVID-19 outcomes in Europe. Biot Sex Differ 11, 29, (2020).

20. Le Bert, N. et al. SARS-CoV-2-specific T cell immunity in cases of COVID-19 and SARS, and uninfected controls. Nature, doi:10.1038/s41586-020-2550-z (2020).

21. Zhang, F. et al. Adaptive immune responses to SARS-CoV-2 infection in severe versus mild individuals. Signal Transduct Target Ther 5, 156, (2020).

22. Ni, L. et al. Detection of SARS-CoV-2-Specific Humoral and Cellular Immunity in COVID-19 Convalescent Individuals. Immunity 52, 971-977 e973, (2020).

23. Lochmanova, A. et al. Quantiferon-CMV test in prediction of cytomegalovirus infection after kidney transplantation. Transplant Proc 42, 3574-3577, (2010).

24. Grijalva C G, Feldstein L R, Talbot H K, et al. Influenza Vaccine Effectiveness for Prevention of Severe Influenza-Associated Illness among Adults in the United States, 2019-2020: A test-negative study. Clin Infect Dis 2021.

25. Polack F P, Thomas S J, Kitchin N, et al. Safety and Efficacy of the BNT162b2 mRNA Covid-19 Vaccine. N Engl J Med 2020; 383: 2603-2615.

26. Voysey M, Clemens S A C, Madhi S A, et al. Safety and efficacy of the ChAdOx1 nCoV-19 vaccine (AZD1222) against SARS-CoV-2: an interim analysis of four randomised controlled trials in Brazil, South Africa, and the UK. Lancet 2021; 397: 99-111.

27. Baden L R, El Sahly H M, Essink B, et al. Efficacy and Safety of the mRNA-1273 SARS-CoV-2 Vaccine. N Engl J Med 2021; 384: 403-416.

28. Garcia-Beltran W F, Lam E C, St Denis K, et al. Multiple SARS-CoV-2 variants escape neutralization by vaccine-induced humoral immunity. Cell 2021; 184: 2372-2383 e2379.

29. Jangra S, Ye C, Rathnasinghe R, et al. SARS-CoV-2 spike E484K mutation reduces antibody neutralisation. Lancet Microbe 2021; 2: e283-e284.

30. Ewer K J, Barrett J R, Belij-Rammerstorfer S, et al. T cell and antibody responses induced by a single dose of ChAdOx1 nCoV-19 (AZD1222) vaccine in a phase 1/2 clinical trial. Nat Med 2021; 27: 270-278.

31. Folegatti P M, Ewer K J, Aley P K, et al. Safety and immunogenicity of the ChAdOx1 nCoV-19 vaccine against SARS-CoV-2: a preliminary report of a phase 1/2, single-blind, randomised controlled trial. Lancet 2020; 396: 467-478.

32. Georg B, Joana B-M, Swantje H, et al. Nature Portfolio 2021.

33. Wang C, Tang J, Song W, Lobashevsky E, Wilson C M, Kaslow R A. HLA and cytokine gene polymorphisms are independently associated with responses to hepatitis B vaccination. Hepatology 2004; 39: 978-988.

34. Gelder C M, Lambkin R, Hart K W, et al. Associations between human leukocyte antigens and nonresponsiveness to influenza vaccine. J Infect Dis 2002; 185: 114-117.

1. A method of determining cell-mediated immune response activity, the method comprising: contacting a sample comprising immune cells capable of producing immune effector molecules following stimulation with a plurality of peptide antigens corresponding to at least one coronavirus protein; and detecting the presence or the level of an immune effector molecule, optionally, wherein the immune effector molecule is selected from one or more of IL-2, IFN-γ, and IL-8.

2. (canceled)

3. (canceled)

4. The method of claim 1, wherein the sample is a whole blood sample.

5.-7. (canceled)

8. The method of claim 1, wherein the plurality of peptide antigens are formulated in a single composition.

9. The method of claim 1, further comprising exposing the sample to an anticoagulant, preferably heparin.

10. (canceled)

11. The method of claim 1, wherein the sample is immunosuppressed or immunodeficient.

12. (canceled)

13. (canceled)

14. The method of claim 1, wherein the plurality of antigens comprises ten or more peptide antigens, and preferably comprises 100 or more peptide antigens.

15. (canceled)

16. The method of claim 1, wherein the antigens are derived from, and therefore cross-reactive with, proteins from a coronavirus (for example, spike glycoprotein, protein 3a, non-structural protein 6, ORF10 protein, ORGF9B protein, non-structural protein 7a, non-structural protein 7b, non-structural protein 8, uncharacterised protein 14, nucleoprotein, membrane protein, and envelope small membrane protein).

17. (canceled)

18. The method of claim 1, wherein the plurality of peptide antigens comprise a set of overlapping peptides that span a region of the at least one coronavirus protein, or span substantially all of the at least one coronavirus protein.

19.-38. (canceled)

39. A composition comprising a biological sample from a subject suspected to have previously been infected with a coronavirus, the sample comprising at least a plurality of peptides antigens that correspond to at least one coronavirus protein and one or more reagents for determining the level of an immune effector molecule.

40. The composition of claim 39, wherein the immune effector molecule is selected from the group comprising IFN-γ, IL-8 and IL-2.

41. (canceled)

42. The composition of claim 39, wherein the plurality of antigens are derived from, and therefore cross-reactive with, at least one coronavirus protein (e.g., spike glycoprotein, protein 3a, non-structural protein 6, ORF10 protein, ORGF9B protein, non-structural protein 7a, non-structural protein 7b, non-structural protein 8, uncharacterised protein 14, nucleoprotein, membrane protein, and envelope small membrane protein).

43.-56. (canceled)

57. The composition of claim 39, wherein the biological sample is a whole blood sample.

58. The composition of claim 57, further comprising an anticoagulant (e.g., heparin).

59. The composition of claim 39, wherein the plurality of peptide antigens comprise at least one set of overlapping peptides that span a region of the at least one coronavirus protein, or that span substantially all of the at least one coronavirus protein.

60. (canceled)

61. The composition of claim 59, wherein the peptides overlap by between 1 and 10 amino acids (i.e., 1, 2, 3, 4, 5, 6, 7, 8, 9, or 10 amino acids).

62. A method of determining the immunocompetency of a vaccinated subject to a virus protein (e.g., a SARS-CoV-2 protein), the method comprising:
contacting a sample optionally, a whole blood sample) from the subject with a plurality of peptide antigens corresponding to at least one virus protein, wherein the sample comprises immune cells capable of producing immune effector molecules following stimulation, optionally, wherein the immune effector molecules are one or both of IL-2 and IFN-γ; and
detecting the presence or the level of an immune effector molecule in the sample;
to thereby determine the immunocompetency of a vaccinated subject to a virus protein.

63.-69. (canceled)

70. The method of claim 62, further comprising exposing the sample to an anticoagulant, preferably heparin.

71. (canceled)

72. The method of claim 62, wherein the subject is immunosuppressed or immunodeficient.

73.-93. (canceled)

94. The method of claim 62, wherein the plurality of peptide antigens comprise a set of overlapping peptides that span a region of the at least one virus protein, or substantially the full length of the at least one virus protein.

95. (canceled)

96. The method of claim 1, wherein the plurality of peptide antigens comprise at least one set of overlapping peptides that span a region of the at least one coronavirus protein or that span substantially all of the at least one coronavirus protein, and wherein the peptides overlap by between 1 and 10 amino acids (i.e., 1, 2, 3, 4, 5, 6, 7, 8, 9, or 10 amino acids).
